SEQUENCE LISTING (36 sequences)

SEQ ID NO: 1 - DNA, 19 bp, artificial sequence
  Synthetic Oligonucleotide
tgctgcatgtgttcctcag

SEQ ID NO: 2 - DNA, 19 bp, artificial sequence
  Synthetic Oligonucleotide
agcttcatcactgactgcc

SEQ ID NO: 3 - DNA, 20 bp, artificial sequence
  Synthetic Oligonucleotide
ggagtgaagaggagtggctg

SEQ ID NO: 4 - DNA, 19 bp, artificial sequence
  Synthetic Oligonucleotide
ttggccacctctgcataac

SEQ ID NO: 5 - DNA, 18 bp, artificial sequence
  Synthetic Oligonucleotide
cgagagcgaggatgatgg

SEQ ID NO: 6 - DNA, 18 bp, artificial sequence
  Synthetic Oligonucleotide
ggagctcctggaccaatg

SEQ ID NO: 7 - DNA, 20 bp, artificial sequence
  Synthetic Oligonucleotide
gatcaggtgatgcagaactc

SEQ ID NO: 8 - DNA, 18 bp, artificial sequence
  Synthetic Oligonucleotide
ggttttacctccattgtg

SEQ ID NO: 9 - DNA, 19 bp, artificial sequence
  Synthetic Oligonucleotide
ccgaaaagggagaagaggc

SEQ ID NO: 10 - DNA, 21 bp, artificial sequence
  Synthetic Oligonucleotide
gatcgttgtctgtattgctgc

SEQ ID NO: 11 - DNA, 26 bp, artificial sequence
  Synthetic Oligonucleotide
gattttccagaccttgtccaagctcc

SEQ ID NO: 12 - DNA, 26 bp, artificial sequence
  Synthetic Oligonucleotide
cactcctcttcactcctttctatggc

SEQ ID NO: 13 - DNA, 25 bp, artificial sequence
  Synthetic Oligonucleotide
cacacagaatgccgctcgctaggag

SEQ ID NO: 14 - DNA, 26 bp, artificial sequence
  Synthetic Oligonucleotide
gtggccaactgagctgcagacttccc

SEQ ID NO: 15 - DNA, 24 bp, artificial sequence
  Synthetic Oligonucleotide
ctgccttggatggtgacatgagtg

SEQ ID NO: 16 - DNA, 26 bp, artificial sequence
  Synthetic Oligonucleotide
gatgtttcctcttaggcctcttctcc

SEQ ID NO: 17 - DNA, 29 bp, artificial sequence
  Synthetic Oligonucleotide
cggaattcatgaagatgagaggggaaaag

SEQ ID NO: 18 - DNA, 30 bp, artificial sequence
  Synthetic Oligonucleotide
cccaagcttttagcacggcttatttgagtc

SEQ ID NO: 19 - DNA, 21 bp, artificial sequence
  Synthetic Oligonucleotide
catgcagacctcattggcctg

SEQ ID NO: 20 - DNA, 27 bp, artificial sequence
  Synthetic Oligonucleotide
gatatcatggcggcgagcgcgctgcga

SEQ ID NO: 21 - DNA, 29 bp, artificial sequence
  Synthetic Oligonucleotide
cggaattcttatttcctctggaaagttag

SEQ ID NO: 22 - DNA, 29 bp, artificial sequence
  Synthetic Oligonucleotide
cggaattcatgtctgaaatcagattcacc

SEQ ID NO: 23 - DNA, 30 bp, artificial sequence
  Synthetic Oligonucleotide
cccaagcttttaactcataccatttgatcc

SEQ ID NO: 24 - DNA, 656 bp, Homo sapiens
ctttggtctagagcaagtgttggcgaactattctgtaaagggctagatagtaaatgtctgaggttcgtaggatcatatggtgtctgtcacaactactcgactctgccattgcagtggaaaagcagccaaggacaatgggtcttcagacctgtagatgggctcttcttggacattctggagtccctgggggaaagggaagtctgaagctcagttggccacctctgcataacaaataccgagagcactgcaagtggagcttggacaaggtctggaaaatcagccttcttctggatgcctccagcctttgctatcatctgcctaggaggttggaagttactcaagcggctcaaggaaaagtccctatttctgttgcagttgatttggagttatgtgatgtgtagcccatcagaagtaagcagaaaactttgtgccgttttctatcctggaaataacacccccgccccctggccccgctgcattatcagccactcctcttcactcctttctatggctccagaagtgaagagctctgaaatgcctaaattgccagatgggaacatctttactgcttggagaaagccacacaggagttacctgacctgccttggatggtgacatgagtgagaaataaacctttgttgggttagactgctaaa

SEQ ID NO: 25 - DNA, 638 bp, Homo sapiens
agagcctctctgcatgagataaggaaaggaatttatgtctgactttggagagattggatggagaagaaagaacagaggaaagagaagacagaaggaagacagaagaaccagaggagcataaccttcagacctgtagatgggctcttcttggacattctggagtccctgggggaaagggaagtctgaagctcagttggccacctctgcataacaaataccgagagcactgcaagtggagcttggacaaggtctggaaaatcagccttcttctggatgcctccagcctttgctatcatctgcctaggaggttggaagttactcaagcggctcaaggaaaagtccctatttctgttgcagttgatttggagttatgtgatgtgtagcccatcagaagtaagcagaaaactttgtgccgttttctatcctggaaataacacccccgccccctggccccgctgcattatcagccactcctcttcactcctttctatggctccagaagtgaagagctctgaaatgcctaaattgccagatgggaacatctttactgcttggagaaagccacacaggagttacctgacctgccttggatggtgacatgagtgagaaataaacctttgttgggttagactgctaaa

SEQ ID NO: 26 - DNA, 776 bp, Homo sapiens
acctcagcctcccaagtagctgggactccaagtgggtgccactacagccatggtgtctttaactgagatatgaagatcacaagcagagaagcttccgagttcagggaggaaatccagagttcggatttgagcatgttaagtttgagatctccgttagacatccacgaaggtcaggtaggcacttgaataaatgagactggaactcagaagaggcttgggctggagcgaaagctgaattgtcagcatatggagaatagaaccttcagacctgtagatgggctcttcttggacattctggagtccctgggggaaagggaagtctgaagctcagttggccacctctgcataacaaataccgagagcactgcaagtggagcttggacaaggtctggaaaatcagccttcttctggatgcctccagcctttgctatcatctgcctaggaggttggaagttactcaagcggctcaaggaaaagtccctatttctgttgcagttgatttggagttatgtgatgtgtagcccatcagaagtaagcagaaaactttgtgccgttttctatcctggaaataacacccccgccccctggccccgctgcattatcagccactcctcttcactcctttctatggctccagaagtgaagagctctgaaatgcctaaattgccagatgggaacatctttactgcttggagaaagccacacaggagttacctgacctgccttggatggtgacatgagtgagaaataaacctttgttgggttagactgctaaa

SEQ ID NO: 27 - DNA, 1019 bp, Homo sapiens
cttttttcatttttttcttcacttttctccagccttcctctctctctctccttccttcctccttgctttctttatatgccttatcttctgagaaggttgaccccagttccggcgatagacggacactaagccccccttgagtgactagaaaggcaggggacataggaacagttaggacataagggaagagtaaagagagggacccctgattttgatttcagggtgccctgtttgtctcatccaatatcctgaagacccaagatggagaccagctacttgtcccttccaaaaagcttccctggccttaatgtcagtgcttgtaggtgatctcagaaacctccctggtgaagaaaacctgaagacggggtcttgctctgttgcctggctggagtgcagtggtgctaacatgactcactgcacccttgacctcctggactcaagtgatcctcccacctcagcctcccaagtagctgggactccaagtgggtgccactacagccaaaccttcagacctgtagatgggctcttcttggacattctggagtccctgggggaaagggaagtctgaagctcagttggccacctctgcataacaaataccgagagcactgcaagtggagcttggacaaggtctggaaaatcagccttcttctggatgcctccagcctttgctatcatctgcctaggaggttggaagttactcaagcggctcaaggaaaagtccctatttctgttgcagttgatttggagttatgtgatgtgtagcccatcagaagtaagcagaaaactttgtgccgttttctatcctggaaataacacccccgccccctggccccgctgcattatcagccactcctcttcactcctttctatggctccagaagtgaagagctctgaaatgcctaaattgccagatgggaacatctttactgcttggagaaagccacacaggagttacctgacctgccttggatggtgacatgagtgagaaataaacctttgttgggttagactgctaa

SEQ ID NO: 28 - DNA, 609 bp, Homo sapiens
gcccaactgtgagggcgcgcgctctcagcagtatataaagcgggacaaccccagaacatcccagttgcaccaggcgatgcaagacacatggcagtctggcctgaagtttcccaaaacaggctgactaggggcctactgcttcccaactaccagctgagggggtccgtcccgaaaagggagaagaggcctaagaggaaacatcaacatctttttactcctagcgagcggcattctgtgtgccttgattgtcttctggaaatatcgctttcagggaaacaatggcgaaatgtcatcagtttcaactgcttttgcactactaagacgcttttctgggttaattagcagcaatacagacaacgatcttttattcaacaacctctctcgagatattttaaataatttctcacactcgaaaaacatgcagaagcgactattggcaaacctgaagagggtggaataccaaatggctgaactggaatattttctagttagcgagggtttgagaggtgcgtcaggtctccagaaattcacctcaaaagcgtacaggatgtaatgccagtggtggaaatcattaaagacactttgagtagaaaaaaaaaaaaaaaaaaa

SEQ ID NO: 29 - protein, 115 aa, homo sapiens
  encoded by (54)..(401) of SEQ ID NO 24
MSEVRRIIWCLSQLLDSAIAVEKQPRTMGLQTCRWALLGHSGVPGGKGSLKLSWPPLHNKYREHCKWSLDKVWKISLLLDASSLCYHLPRRLEVTQAAQGKVPISVAVDLELCDV

SEQ ID NO: 30 - protein, 107 aa, homo sapiens
  encoded by (274)..(597) of SEQ ID NO 25
MPPAFAIICLGGWKLLKRLKEKSLFLLQLIWSYVMCSPSEVSRKLCAVFYPGNNTPAPWPRCIISHSSSLLSMAPEVKSSEMPKLPDGNIFTAWRKPHRSYLTCLGW

SEQ ID NO: 31 - protein, 107 aa, homo sapiens; identical to SEQ ID NO: 30
  encoded by (412)..(735) of SEQ ID NO 26

SEQ ID NO: 32 - protein, 107 aa, homo sapiens; identical to SEQ ID NO: 30
  encoded by (656)..(979) of SEQ ID NO 27

SEQ ID NO: 33 - protein, 88 aa, homo sapiens
  encoded by (287)..(553) of SEQ ID NO 28
MSSVSTAFALLRRFSGLISSNTDNDLLFNNLSRDILNNFSHSKNMQKRLLANLKRVEYQMAELEYFLVSEGLRGASGLQKFTSKAYRM

SEQ ID NO: 34 - DNA, 1831 bp, Homo sapiens
ggtgccgacagggagcttccttggctggcatggcggcgagcgcgctgcgaggcctgccagtggctggcggaggcgagagtagcgagagcgaggatgatggctgggagattgggtatctcgaccggacgtctcagaaattgaaaaggctattacccattgaagaaaagaaagaaaaatttaagaaagcaatgaccatcggagatgtttcattggtccaggagctcctagattctggcattagtgtagattccaactttcagtatggatggactccccttatgtatgctgctagtgttgccaatgcagagctggttcgggtccttttggacagaggtgctaatgcaagctttgagaaggataagcaaagtattttgataactgcatgttctgctcatggctcagaggaacagatcttgaagtgtgtagaactactactttcaagaaatgctgatccaaatgttgcttgtaggagacttatgaccccaatcatgtatgctgctcgagatggtcacacccaggttgttgctctccttgttgctcatggagcagaagttaatacccaggatgagaatggttacactgctttaacgtgggcagcacgtcagggtcataaaaatatagttttgaagttgcttgaacttggagctaataaaatgctacaaaccaaagatggaaagatgccaagtgagattgcaaaaagaaacaaacatcatgagatcttcaacttactttcttttactttaaatccattggaaggaaaacttcaacagctaactaaagaagacactatttgtaaaatattgacaacagattctgatagagaaaaagatcacatttttagttcatatacagcatttggagatctggaagtatttttacatggtcttggacttgaacatatgacagatttactaaaggaaagggatataacgttaagacatcttttgaccatgagggaagatgaatttacaaagaatggaattaccagtaaagaccagcagaaaattctggctgctcttaaagaactacaggtagaagagatacaatttggagagctatctgaagagacaaagttggaaatcagtggtgatgagttcctcaactttcttctcaaattaaataaacagtgtggccatttaataacagctgtacagaatgttattactgagttacctgtaaattctcaaaagataacactggaatgggcttctccccagaattttacttcagtttgtgaagaattggttaataatgttgaagatttgagtgaaaaggtctgcaaactaaaagacctaattcaaaagttgcaaaatgaacgggaaaatgatccaactcatatacaattaagggaagaagtatctacatggaatagtagaattttgaagaggacagctattaccatatgcggattcggttttcttcttttcatttgcaagctaactttccagaggaaataatccaaatatttgttttaagttatatatacacatcttattgaaccatccaaaaatcattgcttttaactgtttgcagcacaatataactttacattctttgccaatattccataggaaaaattttgacaatgtggagtttttttttaaaaagtggtttgttactttgacttataaatacaatttgttatgtttatgttaattgtgattaggtagtaaagtataagtaattttttggtttgatcgtatctaaatcacattcaaaattaaagttatatctaatatctttgacattttttaaagtgtgttcatttgtcagtgaatcgtacaggatttgtaaagtgttcataaattgagctttcttttgtttctttttt

SEQ ID NO: 35 - DNA, 3008 bp, Homo sapiens
atcaggtgatgcagaactctggtaaaggatgttagtcttcagagtaagactgttaaattcagcaaagtctactatcagaagacacgtttttctggttggtgtattactttattcagtgataatatgaaaattactatcttcactgaaggatctactgaataatcattttaactcaactatattgcattcttgaaagcaatcctttcaattgatctactgacttcacaatgtctgaaatcagattcaccaatctcgcttgggatcaagttataacactggatcaagtgttagatgaagtaattccaattcatggaaaggggaatttccccacaatggaggtaaaaccaaaagacatcattcatgttgtgaaagatcaactcatagggcaaggaattattgttaaagatgccagattgaatggttccgtagcaagttacatacttgcaagccacaatggaatcagctataaggatctggacgttatttttggtgttgagcttccaggtaacgaagaatttcaggttgttaaagatgcagttctagactgtctacttgactttttaccaaaagatgtaaagaaggaaaagctctccccagatatcatgaaagacgcttacgtacagaaattggtcaaggtttgcaatgggcatgattgttggagtcttatctccctttcaaataacactgggaagaatttagaactaaaatttgtgagttcactcagacggcagtttgaatttagtgtagattcctttcaaattgttttggatcccatgttagacttctacagtgacaaaaatgccaagctaaccaaagaatcctatcctgttgtggtagctgaaagcatgtatggagacttccaggaagcaatgacacatttgcaacacaagctcatatgtaccaggaaacctgaagagattagaggtggtggccttctgaagtactgcagcttgctggttcatggcttcaagccagcctgtatgtcagaaatcaaaaacctagaacgttatatgtgctctagattctttattgattttcctcatatagaagaacagcaaaagaaaattgaatcatacctccacaaccatttcataggtgaaggaatgaccaagtatgactaccttatgaccttgcatggagttgtgaatgaaagcactgtttgcctcatgagttatgaaagaagacagattctccacctgatcaccatgatggctttgaaagtacttggagaactaaatattctacccaatacacaaaaggtaacttgcttttatcagcctgctccgtactttgcagctgaggcaaggtaccctatttatgtaatacctgagccaccccccgttagcttccagccataccacccactgcaccttcgtggatcaaatggtatgagttaaaaaatacacatgcaaccatagaaactttgatttaattaaacaccatttaaaagcaagtttccaaatgtaaaattcaagatctgtttttatttttgtacagttaccaattattttcaattacagttgatggaatagtagttaccaactatttttgatcaattaagccatcatagtaaatacaatatctataaaccaaccactttaaatgtttttcaaacgttattgactagctataataactttcaaatgttgtgttccccttgaagtgattttcaaatcttttgactacttgtgactttcaaatttgctgtgacatatattagagatgcctgcatctttgactataacataaaaggacagattcatgttttaaaattaagatgtacagtgaagtatcaaattttttatattcagtcagttcctttgttacatttagttctttttttctattgaacaataccatcaataaatgttcagtacttaacagaaatgcctaatttcaaaagcaacagattcagaagacattaaaaccctggacttttcaagcatttttttttgacaattaaattgggttggatacaaaaatcctactatagtttaaaggaatactggaaaaaaatggttctggaaagccctggacattagtaatttgtcatctatataataaaacatttagttaatttgtggatttgaataacaataccatacagctgtgcaactactactgagatcagatacagggttttttgcccctcaaaataagttaaaacgaatgaaaatgaccagctctgaatgtgaaagctttctatcatcatctactacaaagagactctaaatggcaaacaggaaaaaaacaggcagaggtttatagccatagcatttaccattttatggcttatttgaaaaacatctttatgttgagaaacattccatttcagtaagttaaaatattttcattgtaactaaatgaagcaggtttttcattttgcctttaccaaagatcattttctaacatgccactcaagtgcctcttcgtgtgaatttttgcgtaacactataatttattcaacaactgtatacctttacagtatatctaaatatatacttactacatcgagtatactgctagaaaaatctaccagtgggattaaaaatgtattttcttcccataatgaaaataattcatgaccaatattacctgaagtgtcagaagaggagctgtcttaaattaatatggatcaggccattggaaaacttcagtatttccacttggttatgattttttgtgagtttccatttcagtttttattgaccatcacgttttctatcacaacatgacctgacagttgtatcccgataatttgccagttatagactgtgcttatcttcactgtattacaatattggacagaagtacaccaaatacggcattttcaaataaccaatatttctgagatgttttataccacagcacaagtggccatcagtatatttttaaagaattatatatcacagaaccctgaggctttaaaacactatggacttaccactttcttgacaaagaatttgtaattacaataaaatatttagaaatgaaaaaaaaaaaaaaaa

SEQ ID NO: 36 - DNA, 3250 bp, Homo sapiens
agttccagagacagtagttatctcgggccccaacatcgcgaggtctagggaaaaatgctaccactcctcccccctacttccgctgccacgctgcctccatgcctgcctcccctctctgccacgcccccaccacgcgctttgcactctggggcccacgcacttccctgaagagtctagaagctgctcctcatttccagactttccgggcgcccaccaggctcccgggctctcaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggtttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaatgccaaagtgcttttcaatctagtaaatctagagggttgttttgtcttagccacaagaattccgaggtcttgaccctgatgatcaacctgcctcccctccatagtcttgttggagaagcccagagagaatgggactccaactaagggaacctgaaatcaactcaatggaggcacttcagagctaaaataattatggcttccttgcttaataaacattttcgttcactgcaa

(19) United States
(12) Patent Application Publication
(10) Pub. No.: US 2011/0311539 A1
(43) Pub. Date: Dec. 22, 2011

(54) DIAGNOSIS AND TREATMENT OF CANCER USING CANCER-TESTIS ANTIGENS

(75) Inventors: Fabiana Bettoni, Sao Paulo (BR); Anamaria Aranha Camargo, Sao Paulo (BR)

(73) Assignee: Ludwig Institute for Cancer Research Ltd, New York, NY (US)

(21) Appl. No.: 12/678,558

(22) PCT Filed: Sep. 16, 2008
(86) PCT No.: PCT/US08/10777

§ 371 Date: Sep. 23, 2010

Related U.S. Application Data
(60) Provisional application No. 60/994,237, filed on Sep. 17, 2007.

(51) Int. Cl. A61K 39/395; C12N 1/21; C12N 5/10; C12N 1/19; C12Q 1/68; A61P 37/04; C07H 21/00; C07H 21/04; A61K 39/00; C07K 14/47; C07K 16/30; A61P 35/00; C12N 15/63; G01N 33/566

(52) U.S. Cl. 424/139.1; 435/320.1; 435/252.33; 435/362; 435/365; 435/254.2; 435/348; 435/366; 435/354; 435/352; 435/325; 435/363; 435/6.11; 435/7.92; 435/6.12; 536/24.33; 536/23.5; 424/277.1; 424/185.1; 530/350; 530/387.3; 530/387.9; 530/324

(57) ABSTRACT

The invention relates to CT polypeptides and the nucleic acid molecules that encode them. The invention further relates to the use of the nucleic acid molecules, polypeptides and fragments thereof in methods and compositions for the diagnosis, prognosis and treatment of diseases, such as cancer. More specifically, the invention relates to the discovery of a novel cancer/testis (CT) antigens CT1.1 (CTSP-5), CT1.11 (CTSP-6), CT1.19 (CTSP-7), CT1.26 (CTSP-8), and CT1.29 (CTSP-9).

### RELATED APPLICATIONS

This application claims the benefit under 35 U.S.C. §119(e) of U.S. provisional application 60/994,237, filed Sep. 17, 2007, the disclosure of which is incorporated herein by reference.

### FIELD OF THE INVENTION

The invention relates to a family of cancer-testis antigens and the nucleic acid molecules that encode them. The invention further relates to the use of the nucleic acid molecules, polypeptides and fragments thereof in methods and compositions for the diagnosis, prognosis and treatment of diseases, such as cancer. More specifically, the invention relates to the discovery of novel cancer/testis (CT) antigens, CT1.1 (CTSP-5), CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9).

### BACKGROUND OF THE INVENTION

CT antigens are predominantly expressed in normal gametogenic tissues as well as in different histological types of tumors (Scanlan et al., 2002, Immunol Rev. 188:22-32. Review; Scanlan et al., 2004, Cancer Immun. 4:1. Review; Simpson et al., 2005, Nat Rev Cancer. 5(8):615-25. Review; Zendman et al., 2003, J Cell Physiol. 194(3):272-88. Review). In testis, CT antigens are expressed exclusively in cells of the germ cell lineage, although there is a marked variation in the protein expression pattern during different stages of sperm development. Likewise, a heterogeneous expression is also observed in tumors (Scanlan et al., 2002, Immunol Rev. 188:22-32. Review; Zendman et al., 2003, J Cell Physiol. 194(3):272-88. Review). Methylation status of the promoter region seems to be the main, but not the only regulator of their specific expression pattern (Scanlan et al., 2002, Immunol Rev. 188:22-32. Review; Simpson et al., 2005, Nat Rev Cancer. 5(8):615-25. Review; Zendman et al., 2003, J Cell Physiol. 194(3):272-88. Review). Most CT antigens have no defined biological function but their involvement in signaling, transcription, translation and chromosomal recombination has been proposed (Simpson et al., 2005, Nat Rev Cancer. 5(8):615-25. Review; Zendman et al., 2003, J Cell Physiol. 194(3):272-88. Review). It has also been proposed that the aberrant expression of CT antigens in tumors recapitulates portions of the germline gene expression programme and is related to some characteristics of the neoplastic phenotype such as immortality, invasiveness, immune evasion and metastatic capacity (Simpson et al., 2005, Nat Rev Cancer. 5(8):615-25. Review; Old, L. J., 2001, Cancer Immun. 1: 1).

Due to their restricted expression pattern, CT antigens are considered to be ideal targets for cancer immunotherapy (Scanlan et al., 2002, Immunol Rev. 188:22-32. Review; Bodey, 2002, Expert Opin Biol Ther. 2(6):577-84. Review). Indeed, a small subset of patients immunized with the known CT antigens MAGE-A and NY-ESO-1 have shown clinical benefits following immunization (Chen et al., 2004, Proc. Natl. Acad. Sci. U.S.A. 101: 9363-9368; Davis et al., 2004, Proc. Natl. Acad. Sci. U.S.A. 101:10697-10702; Jager et al., 2000, Proc. Natl. Acad. Sci. U.S.A. 97: 12198-12203; Marchand et al., 1999, Int. J. Cancer. 80: 219-230). However, because CT antigens are expressed in only a small subset of human tumors and in only a fraction of cases of a given tumor type, the identification of additional CT antigens is important for improving current immunotherapy protocols.

The identification of additional CT antigens and other genes having a tumor-associated expression profile is needed for the development of additional therapeutics and diagnostics to permit effective treatment and diagnosis of a broader group of cancer patients.

### SUMMARY OF THE INVENTION

A computational approach based on expression data from expressed sequence tags (ESTs) was used to identify novel Cancer-Testis Antigens (CTs). Expressed sequences (mRNA and ESTs) were aligned against the human genome sequence, allowing the clustering of the sequences derived from the same gene. Considering the tissue of origin of the ESTs in a cluster, it was possible to define in silico the expression pattern of the corresponding gene and to select novel CT antigen candidates. A total of 1255 clusters composed of spliced ESTs derived from testis and/or tumoral cDNA libraries were identified and 70 of them were selected for experimental validation of their expression pattern. The experimental validation of the expression pattern was carried out by RT-PCR in 21 normal tissues and 17 tumor cell lines. Five CT antigen candidates were identified (CT1.1 (CTSP-5), CT1.11 (CTSP-6) splice variants: CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9)) that were expressed mainly in testis among normal tissues and frequently expressed in different types of tumors. The discovery of these 5 novel CTs, and the nucleic acid molecules, polypeptides and fragments thereof provided, are useful in methods and compositions for the diagnosis of cancer, the monitoring of disease progression, and for the treatment of cancer.

According to one aspect of the invention, an isolated nucleic acid molecule is provided selected from the group consisting of: (a) complements of nucleic acid molecules that hybridize under high stringency conditions to a second nucleic acid molecule comprising a nucleotide sequence set forth as any of SEQ ID NOs: 24-28, (b) nucleic acid molecules that differ from the nucleic acid molecules of (a) in codon sequence due to the degeneracy of the genetic code, and (c) full-length complements of (a) or (b).

In some embodiments, the isolated nucleic acid molecule comprises a nucleotide sequence set forth as any of SEQ ID NOs: 24-28, while in other embodiments the isolated nucleic acid molecule consists of a nucleotide sequence set forth as any of SEQ ID NOs: 24-28.

In further embodiments isolated nucleic acid molecule comprises a nucleotide sequence set forth as any of SEQ ID NOs: 24-28, a protein-coding portion thereof, or an alternatively spliced product thereof. in other embodiments the nucleic acid molecule consists of a nucleotide sequence set forth as any of SEQ ID NO: 24-28, a protein-coding portion thereof, or an alternatively spliced product thereof.

In some embodiments the isolated nucleic acid molecule is at least about 90% identical to a nucleotide sequence set forth as any of SEQ ID NOs: 24-28 or a full-length complement thereof, while in other embodiments the isolated nucleic acid molecule is at least about 95%, 96%, 97%, 98%, 99% identical.

According to some aspects of the invention, compositions are provided that include any of the foregoing isolated nucleic acid molecules and a carrier, or any of the foregoing isolated nucleic acid molecules attached to a solid substrate.

According to some aspects of the invention, kits are provided comprising one or more nucleic acid molecules that hybridize under high stringency conditions to a nucleic acid molecules that encodes CT1.11 (CTSP-6), CT1.19 (CTSP-7), CT1.26 (CTSP-8), and CT1.29 (CTSP-9). In certain embodiments kits are provided additionally comprising a nucleic acid molecule that hybridizes under high stringency conditions to a nucleic acid molecules that encodes CT1.1 (CTSP-5).

In some embodiments the one or more nucleic acid molecules are detectably labeled. In other embodiments the one or more nucleic acid molecules consist of a first primer and a second primer, wherein the first primer and the second primer are constructed and arranged to selectively amplify at least a portion of a nucleic acid molecule encoding CT1.11 (CTSP-6), CT1.19 (CTSP-7), CT1.26 (CTSP-8), and CT1.29 (CTSP-9).

In another embodiments the kits may further comprise an additional primer pair, a first primer and a second primer, to selectively amplify at least a portion of a nucleic acid molecule encoding CT1.1 (CTSP-5). In yet another embodiments the one or more nucleic acids are bound to a solid substrate.

According to another aspect of the invention, expression vectors including any of the forgoing isolated nucleic acid molecules, operably linked to a promoter, are provided. Additionally provided are isolated host cell transformed or transfected with the expression vectors. In some embodiments, the host cells express a MHC molecule, preferably recombinantly. In some embodiments the host cell is a dendritic cell.

Also provided in accordance with the invention are compositions that include the foregoing isolated host cells and a carrier.

According to another aspect of the invention, isolated polypeptides encoded by any of the foregoing isolated nucleic acid molecules are provided, as are fragments of the polypeptides that are at least eight amino acids in length. In some embodiments, the isolated polypeptide includes, or preferably consists of, an amino acid sequence set forth as any of SEQ ID NOs: 29-33, as well as an isolated polypeptide encoded by the CT1.19 (CTSP-7) (SEQ ID NO. 34) or CT1.26 (CTSP-8) (SEQ ID NO. 35) gene, or a fragment thereof that is at least eight amino acids in length.

According to another aspect of the invention, compositions including any of the foregoing isolated polypeptides and a carrier are provided; preferably the compositions also include an adjuvant. Also provided are compositions including any of the foregoing isolated polypeptides attached to a solid substrate.

In some embodiments, the compositions further include at least one additional cancer-testis antigen polypeptide, preferably a CT1.1 (CTSP-5) polypeptide (such as encoded by SEQ ID NO. 36), preferably also containing a carrier and/or an adjuvant.

According to yet another aspect of the invention, isolated antibodies or antigen-binding fragments thereof are provided that selectively bind to the foregoing isolated polypeptides. In some embodiments, the antibody is a monoclonal antibody, a human antibody, a domain antibody, a humanized antibody, a single chain antibody or a chimeric antibody. In other embodiments, the isolated antigen-binding fragment thereof is a F(ab′)₂, Fab, Fd, or Fv fragment. Also provided are compositions including any of the foregoing isolated antibodies or antigen-binding fragments and a carrier, or attached to a solid substrate. Kits including the isolated antibodies or antigen-binding fragments also are provided. In addition the aforementioned compositions and kits may further comprise an isolated antibody or antigen-binding fragment that selectively binds to an isolated CT1.1 (CTSP-5) polypeptide (such as encoded by the nucleic acid SEQ ID NO. 36).

According to a tenth aspect of the invention, methods of diagnosing cancer in a subject are provided. The methods include obtaining a biological sample from the subject, and determining the presence in the biological sample of an antibody that binds specifically to one or more polypeptides encoded by a nucleotide sequence set forth as any of SEQ ID NO: 24-28 or encoded by the CT1.19 (CTSP-7) (SEQ ID NO. 34) or CT1.26 (CTSP-8) (SEQ ID NO. 35) gene. The presence of the antibody is indicative of the subject having cancer.

In some embodiments, the step of determining the presence of the antibody includes contacting the biological sample with one or more polypeptides encoded by a nucleic acid molecule comprising (1) a nucleotide sequence set forth as any of SEQ ID NOs: 24-28 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1), and determining the binding of the antibody to the polypeptide. In certain embodiments, the polypeptide includes an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length. Preferably the polypeptide is produced recombinantly and/or is bound to a substrate.

In other embodiments, the step of determining the binding of the antibody with the polypeptide is performed with an ELISA-based method.

In preferred embodiments of the methods, the biological sample is blood or serum.

In certain embodiments the aforementioned methods further comprise determining the presence in the biological sample of an additional antibody that binds specifically to the CT1.1 (CTSP-5) polypeptide.

According to another aspect of the invention, methods for diagnosing cancer in a subject are provided. The methods include obtaining a biological sample from a subject, and determining the expression in the biological sample of a polypeptide or a nucleic acid molecule that encodes the polypeptide, wherein the nucleic acid molecule includes (1) a nucleotide sequence set forth as any of SEQ ID NOs:24-28 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1). The expression in the biological sample of the polypeptide or the nucleic acid molecule that encodes it is indicative of the subject having cancer.

In some embodiments, the polypeptide includes an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length.

In other embodiments, the step of determining the expression of the polypeptide or the nucleic acid molecule that encodes the polypeptide includes contacting the biological sample with an agent that selectively binds to the polypeptide or the nucleic acid molecule that encodes the polypeptide.

In some embodiments, the agent is a nucleic acid probe or a nucleic acid primer. Optionally, the expression of the nucleic acid molecule is determined by nucleic acid hybridization using the nucleic acid probe or nucleic acid amplification using the nucleic acid primer. Preferably the nucleic acid amplification is real-time RT-PCR or RT-PCR. Preferably the nucleic acid hybridization is performed using a nucleic acid microarray containing the nucleic acid probe.

In some embodiments, the agent is a polypeptide, preferably an antibody or antigen-binding fragment thereof, more preferably a monoclonal antibody or a F(ab′)₂, Fab, Fd, or Fv fragment. In certain embodiments, the antibody or antigen-binding fragment is labeled with a detectable label, preferably a fluorescent or radioactive label.

In preferred embodiments of the methods, the sample comprises tissue, cells, and/or blood.

In certain embodiments the aforementioned methods further comprise determining the expression in the biological sample of a CT1.1 (CTSP-5) polypeptide or a CT1.1 (CTSP-5) nucleic acid molecule (SEQ ID NO. 36) that encodes the polypeptide.

According to another aspect of the invention, methods for determining onset, progression, or regression of cancer in a subject are provided. The methods include obtaining from a subject a first biological sample at a first time, determining the expression in the first sample of a polypeptide or a nucleic acid molecule that encodes the polypeptide, wherein the nucleic acid molecule includes (1) a nucleotide sequence set forth as any of SEQ ID NOs:24-28 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1), obtaining from the subject a second biological sample at a second time subsequent to the first time, determining the expression in the second sample of the polypeptide or the nucleic acid molecule that encodes the polypeptide, and comparing the expression in the first sample to the expression in the second sample as a determination of the onset, progression, or regression of the cancer. An increase in expression in the second sample compared to the first sample is indicative of onset or progression of the cancer, and a decrease in the expression in the second sample compared to the first sample is indicative of regression of the cancer.

In certain embodiments, the polypeptide includes an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length.

In other embodiments, the step of determining the expression of the polypeptide or the nucleic acid molecule that encodes the polypeptide includes contacting first biological sample and the second biological sample with an agent that selectively binds to the polypeptide or the nucleic acid molecule that encodes the polypeptide.

In some embodiments, the agent is a nucleic acid probe or a nucleic acid primer. Optionally, the expression of the nucleic acid molecule is determined by nucleic acid hybridization using the nucleic acid probe or nucleic acid amplification using the nucleic acid primer. Preferably the nucleic acid amplification is real-time RT-PCR or RT-PCR. Preferably the nucleic acid hybridization is performed using a nucleic acid microarray containing the nucleic acid probe.

In some embodiments, the agent is a polypeptide, preferably an antibody or antigen-binding fragment thereof, more preferably a monoclonal antibody or a F(ab′)₂, Fab, Fd, or Fv fragment. In certain embodiments, the antibody or antigen-binding fragment is labeled with a detectable label, preferably a fluorescent or radioactive label.

In preferred embodiments of the methods, the sample comprises tissue, cells, and/or blood.

In certain embodiments any of the aforementioned methods comprise determining the expression in the first and second sample of a CT1.1 (CTSP-5) polypeptide or a CT1.1 (CTSP-5) nucleic acid molecule (SEQ ID NO. 36) that encodes the polypeptide.

According to another aspect of the invention, methods for treating cancer in a subject are provided. The methods include administering to the subject an agent that stimulates an immune response to a polypeptide encoded by a nucleic acid molecule comprising a nucleotide sequence that is at least 90% identical to the nucleotide sequence set forth as any of SEQ ID NOs:24-28 or a nucleotide sequence of CT1.19 (CTSP-7) (SEQ ID NO. 34) or CT1.26 (CTSP-8) (SEQ ID NO. 35).

In some embodiments, the nucleic acid molecule includes the nucleotide sequence set forth as any of SEQ ID NOs:24-28. In other embodiments, the polypeptide includes an amino acid sequence set forth as any of SEQ ID NOs:29-33, or a fragment thereof that is at least eight amino acids in length.

In some embodiments, the agent that stimulates the immune response is a nucleic acid that encodes the polypeptide operably linked to a promoter; the polypeptide; a cell that expresses the polypeptide, preferably a cell that also expresses a MHC molecule; a peptide fragment of the polypeptide; or a complex of a peptide fragment of the polypeptide and a MHC molecule. Optionally the agent further comprises an adjuvant or a cytokine.

In some embodiments, the immune response elicited by an agent or agents according to the invention is a B cell response. In some embodiments, the immune response elicited by an agent or agents according to the invention is a T cell response, preferably a CD4+ T cell and/or CD8+ T cell response.

In certain embodiments the aforementioned methods further comprise administering to the subject an agent that stimulates an immune response to a polypeptide encoded by a nucleic acid molecule comprising a nucleotide sequence that is at least 90% identical to the nucleotide sequence of CT1.1 (CTSP-5) (SEQ ID NO. 36).

According to yet another aspect of the invention, methods for treating cancer in a subject are provided. The methods include administering to a subject an effective amount of an antibody or antigen-binding fragment thereof that specifically binds to a polypeptide that comprises an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a peptide fragment thereof, or a complex of the peptide fragment and a MHC or HLA molecule.

In some embodiments, the antibody is a monoclonal antibody, preferably a chimeric, human, or humanized antibody, or a single chain antibody. In other embodiments, the antigen-binding fragment is a F(ab′)₂, Fab, Fd, or Fv fragment.

In still other embodiments, the antibody or antigen-binding fragment thereof is bound to a cytotoxic agent, preferably one selected from the group consisting of: calicheamicin, esperamicin, methotrexate, doxorubicin, melphalan, chlorambucil, ARA-C, vindesine, mitomycin C, cisplatinum, etoposide, bleomycin and 5-fluorouracil. In further embodiments, the cytotoxic agent is a radioisotope, preferably one that emits α radiation, β radiation or γ radiation. In particular embodiments, the radioisotope is selected from the group consisting of ^{225A}, ²¹¹At, ²¹²Bi, ²¹³Bi, ₁₈₆Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Bo, ⁶⁴Cu, ²¹²Pb, ²²⁴Ra and ²²³Ra.

In certain embodiments the aforementioned methods further comprise administering to a subject an effective amount of an antibody or antigen-binding fragment thereof that specifically binds to a polypeptide that comprises an amino acid sequence corresponding to the CT1.1 (CTSP-5) gene (SEQ ID NO. 36), or a peptide fragment thereof, or a complex of the peptide fragment and a MHC or HLA molecule.

The invention also involves the use of the genes, gene products, fragments thereof, agents which bind thereto, and other compositions and molecules described herein in the preparation of medicaments. A particular medicament is for treating cancer. These and other aspects of the invention will be described in further detail in connection with the detailed description of the invention.

### BRIEF DESCRIPTION OF THE DRAWINGS

FIG. 1 depicts immunoblots detecting antibodies against CT recombinant proteins in plasma samples from cancer patients. Lanes 1 and 2: plasma samples from cancer patients; lanes 3 and 4: plasma samples from healthy donors; lane 5 anti-His-tag antibody used as a positive control.
a) CT1.1 (CTSP-5) recombinant protein and plasma samples from uterus cancer patients.
b) CT1.19 (CTSP-7) recombinant protein and plasma samples from uterus cancer patients.
c) CT1.26 (CTSP-8) recombinant protein and plasma samples from lung cancer patients.

FIG. 2 depicts mRNA expression pattern of the five candidates (CT1.1/CTSP-5, CT1.11/CTSP-6, CT1.19/CTSP-7, CT1.26/CTSP-8, CT1.29/CTSP-9) in normal tissues and tumor cell lines. Southern blot of RT-PCR products amplified with each candidate specific primers. (A) Normal cDNA samples used were as follows. Lanes: 1, brain; 2, breast; 3, lung; 4, prostate; 5, small intestine; 6, cerebellum; 7, colon; 8, fetal brain; 9, fetal liver; 10, heart; 11, kidney; 12, placenta; 13, salivary gland; 14, skeletal muscle; 15, spinal cord; 16, spleen; 17, stomach; 18, thymus; 19, trachea; 20, uterus; 21, testis. GAPDH amplification was used as positive control for cDNA synthesis. (B) cDNA samples from tumor cell lines used were: 1, HL-60; 2, SCABER; 3, A172; 4, T98G; 5, MCF-7; 6, MDA-MB-436; 7, SW-480; 8, FADu; 9, H1155; 10, H358; 11, A2058; 12, SKmel-25; 13, SAOS-2; 14, Du145; 15, PC3; 16, Caski; 17, HeLa; 18, no cDNA-negative control. GAPDH amplification was used as positive control for cDNA synthesis.

FIG. 3 depicts a survival curve of 50 glioblastoma patients according to the presence of an anti-CT1.1 humoral immune response.

### BRIEF DESCRIPTION OF THE SEQUENCES

SEQ ID NO:1 is the nucleotide sequence of the CT1.1 (CTSP-5)F primer.

SEQ ID NO:2 is the nucleotide sequence of the CT1.1 (CTSP-5)R primer.

SEQ ID NO:3 is the nucleotide sequence of the CT1.11 (CTSP-6)F primer.

SEQ ID NO:4 is the nucleotide sequence of the CT1.11 (CTSP-6)R primer.

SEQ ID NO:5 is the nucleotide sequence of the CT1.19 (CTSP-7)F primer.

SEQ ID NO:6 is the nucleotide sequence of the CT1.19 (CTSP-7)R primer.

SEQ ID NO:7 is the nucleotide sequence of the CT1.26 (CTSP-8)F primer.

SEQ ID NO:8 is the nucleotide sequence of the CT1.26 (CTSP-8)R primer.

SEQ ID NO:9 is the nucleotide sequence of the CT1.29 (CTSP-9)F primer.

SEQ ID NO:10 is the nucleotide sequence of the CT1.29 (CTSP-9)R primer.

SEQ ID NO:11 is the nucleotide sequence of the CT1.11 (CTSP-6) 5′-RACE-F primer.

SEQ ID NO:12 is the nucleotide sequence of the CT1.11 (CTSP-6) 3′-RACE-R primer.

SEQ ID NO:13 is the nucleotide sequence of the CT1.11 (CTSP-6) 5′-RACE-FN primer.

SEQ ID NO:14 is the nucleotide sequence of the CT1.11 (CTSP-6) 3′-RACE-RN primer.

SEQ ID NO:15 is the nucleotide sequence of the CT1.29 (CTSP-9) 3′-RACE-R primer.

SEQ ID NO:16 is the nucleotide sequence of the CT1.29 (CTSP-9) 3′-RACE-RN primer.

SEQ ID NO:17 is the nucleotide sequence of the CT1.1 (CTSP-5) PTN101F primer.

SEQ ID NO:18 is the nucleotide sequence of the CT1.1 (CTSP-5) PTN101R primer.

SEQ ID NO:19 is the nucleotide sequence of the CT1.1 (CTSP-5) PROT101R primer.

SEQ ID NO:20 is the nucleotide sequence of the CT1.19 (CTSP-7) PTN802F32 primer.

SEQ ID NO:21 is the nucleotide sequence of the CT1.19 (CTSP-7) PTN802R32 primer.

SEQ ID NO:22 is the nucleotide sequence of the CT1.26 (CTSP-8) PTN809F primer.

SEQ ID NO:23 is the nucleotide sequence of the CT1.26 (CTSP-8) PTN809R primer.

SEQ ID NO:24 is the nucleotide sequence of the CT1.11a (CTSP-6.1).

SEQ ID NO:25 is the nucleotide sequence of the CT1.11b (CTSP-6.2).

SEQ ID NO:26 is the nucleotide sequence of the CT1.11c (CTSP-6.3).

SEQ ID NO:27 is the nucleotide sequence of the CT1.11d (CTSP-6.4).

SEQ ID NO:28 is the nucleotide sequence of the CT1.29 (CTSP-9).

SEQ ID NO:29 is the amino acid sequence of polypeptide encoded by the open reading frame of CT1.11a (CTSP-6.1) (115 aa).

SEQ ID NO:30 is the amino acid sequence of polypeptide encoded by the open reading frame of CT1.11b (CTSP-6.2) (107 aa).

SEQ ID NO:31 is the amino acid sequence of polypeptide encoded by the open reading frame of CT1.11c (CTSP-6.3) (107 aa).

SEQ ID NO:32 is the amino acid sequence of polypeptide encoded by the open reading frame of CT1.11d (CTSP-6.4) (107 aa).

SEQ ID NO:33 is the amino acid sequence of polypeptide encoded by the open reading frame of CT1.29 (CTSP-9) (88 aa).

SEQ ID NO:34 is the nucleotide sequence of CT1.19 (CTSP-7) (Accession No. AF461259).

SEQ ID NO:35 is the nucleotide sequence of CT1.26 (CTSP-8) (Accession No. BC028710).

SEQ ID NO:36 is the nucleotide sequence of CT1.1 (CTSP-5) (Accession No. NM_{—}173493).

### DETAILED DESCRIPTION OF THE INVENTION

The invention relates to CT1.1 (CTSP-5), CT1.11 a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9) antigen polypeptides provided herein and the nucleic acid molecules that encode them. The sequences of the CTs of the invention are identified as follows: CT1.1 (CTSP-5) (NM173493, BC040301, PASD1, gi|27735094|), CT1.11 (CTSP-6) (AI652043), CT1.11a (CTSP-6.1) (EF537578), CT1.11b (CTSP-6.2) (EF537579), CT1.11c (CTSP-6.3) (EF537580), CT1.11d (CTSP-6.4) (EF537581), CT1.19 (CTSP-7) (AF461259, GASZ, ASZ1, gi|8389975|), CT1.26 (CTSP-8) (BC028710, FAM46D, gi|34192069|) and CT1.29 (CTSP-9) (AA451827, EF537582).

The invention further relates to the use of the nucleic acid molecules, polypeptides and fragments thereof in methods and compositions for the diagnosis and treatment of cancer.

As used herein, the terms “CT nucleic acid” and “CT polypeptide”, and the like refer to a family of nucleic acids and polypeptides that are at least about 90%, 95%, 96%, 97%, 98%, or 99% identical to the sequences of SEQ ID NOS: 24-28 (nucleic acid sequence of CT1.11a (CTSP-6.1), CT1.11b (CTSP-6.2), CT1.11c (CTSP-6.3), CT1.11d (CTSP-6.4) and CT1.29 (CTSP-9)) or any of SEQ ID NOS: 29-33 (polypeptide sequence of CT1.11a (CTSP-6.1), CT1.11b (CTSP-6.2), CT1.11c (CTSP-6.3), CT1.11d (CTSP-6.4) and CT1.29 (CTSP-9)), as well as nucleic acid sequences and polynucleotide sequences related to CT1.1 (CTSP-5), CT1.19 (CTSP-7), and CT1.26 (CTSP-8).

The polypeptides elicit specific immune responses as is shown in the Examples below, and thus include CT polypeptides (including proteins) and fragments of CT polypeptides that are recognized by the immune system (e.g., by antibodies and/or T lymphocytes).

In part, the invention relates to CT polypeptides CT1.11 (CTSP-6), CT1.19 (CTSP-7), CT1.26 (CTSP-8), and CT1.29 (CTSP-9) as well as the nucleic acid molecules that encode the CT polypeptides. As used herein, the “nucleic acid molecules that encode” means the nucleic acid molecules that code for the CT polypeptides or fragments thereof, particularly immunogenic fragments. These nucleic acid molecules may be DNA or may be RNA (e.g., mRNA). The CT nucleic acid molecules of the invention also encompass variants of the nucleic acid molecules described herein. These variants may be splice variants, some of which are described herein for CT1.11 (CTSP-6), or allelic variants. Variants of the nucleic acid molecules of the invention are intended to include homologs and alleles which are described further below. Further, as used herein, the term “CT molecules” includes CT polypeptides and fragments thereof as well as CT nucleic acids and fragments (such as exon sequences). In all embodiments, human CT polypeptides and the nucleic acid molecules that encode them are preferred.

In one aspect, the invention provides an isolated nucleic acid molecule selected from the group consisting of: (a) complements of nucleic acid molecules that hybridize under high stringency conditions to a second nucleic acid molecule comprising a nucleotide sequence set forth as any of SEQ ID NOs: 24-28, (b) nucleic acid molecules that differ from the nucleic acid molecules of (a) in codon sequence due to the degeneracy of the genetic code, and (c) full-length complements of (a) or (b).

As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a small percentage of the material in the cell in which it resides. Such a nucleic acid is isolated, however, as the term is used herein because it is readily manipulable by standard techniques known to those of ordinary skill in the art.

The CT nucleic acid molecules of the invention (CT1.1 (CTSP-5), CT1.11 (CTSP-6) splice variants: CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9)) also encompass homologs and alleles which can be identified by conventional techniques. Identification of human and other organisms' homologs of CT polypeptides will be familiar to those of skill in the art. In general, nucleic acid hybridization is a suitable method for identification of homologous sequences of another species (e.g., human, cow, sheep, dog, rat, mouse), which correspond to a known sequence. Standard nucleic acid hybridization procedures can be used to identify related nucleic acid sequences of selected percent identity. For example, one can construct a library of cDNAs reverse transcribed from the mRNA of a selected tissue and use the CT nucleic acid molecules identified herein to screen the library for related nucleotide sequences. The screening preferably is performed using high-stringency conditions to identify those sequences that are closely related by sequence identity. Nucleic acids so identified can be translated into polypeptides and the polypeptides can be tested for activity.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1× SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of the CT nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

In general, homologs and alleles typically will share at least 90% nucleotide identity and/or amino acid identity to the sequences of CT nucleic acids and polypeptides, respectively, in some instances will share at least 95% nucleotide identity and/or amino acid identity, in other instances will share at least 97% nucleotide identity and/or amino acid identity, in other instances will share at least 98% nucleotide identity and/or amino acid identity, and in other instances will share at least 99% nucleotide identity and/or amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using a number of sequence analysis software programs, such as the MacVector sequence analysis software (Accelrys Software Inc., San Diego, Calif.). Watson-Crick complements of the foregoing nucleic acids also are embraced by the invention.

In another aspect of the invention, unique fragments are provided which include unique fragments of the nucleotide sequences of the invention and complements thereof. The invention, in a preferred embodiment, provides unique fragments of SEQ ID NO:24-28 and CT1.19 (CTSP-7) and CT1.26 (CTSP-8) and complements thereof. A unique fragment is one that is a ‘signature’ for the larger nucleic acid. It, for example, is long enough to assure that its precise sequence is not found in molecules outside of the nucleic acid molecules that encode the CT polypeptides defined above. Those of ordinary skill in the art may apply no more than routine procedures to determine if a fragment is unique within the human genome. In some instances the unique fragment is at least about 5, 6, 7, 8, 9, 10, 11, 12, 13, 14, 15, 16, 17, 18, 19, 20, 25, 30, 35, 40, 45, 50, 75, or 100 nucleotides in length.

Unique fragments can be used as probes in Southern blot assays to identify such nucleic acid molecules, or can be used as probes in amplification assays such as those employing the polymerase chain reaction (PCR), including, but not limited to RT-PCR and RT-real-time PCR. As known to those skilled in the art, large probes such as 200 nucleotides or more are preferred for certain uses such as Southern blots, while smaller fragments will be preferred for uses such as PCR. Standard instrumentation known to those skilled in the art may be used for PCR, e.g. Johnson et al, U.S. Pat. No. 5,038,852 (computer-controlled thermal cycler); Wittwer et al, Nucleic Acids Research, 17: 4353-4357 (1989) (capillary tube PCR); Hallsby, U.S. Pat. No. 5,187,084 (air-based temperature control); Garner et al, Biotechniques, 14: 112-115 (1993) (high-throughput PCR in 864-well plates); Wilding et al, International application No. PCT/US93/04039 (PCR in micro-machined structures); Schnipelsky et al, European patent application No. 90301061.9 (publ. No. 0381501 A2) (disposable, single use PCR device), and the like.

Unique fragments also can be used to produce fusion proteins for generating antibodies or determining binding of the polypeptide fragments, or for generating immunoassay components. Techniques for making fusion proteins are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

Likewise, unique fragments can be employed to produce nonfused fragments of the CT polypeptides useful, for example, in the preparation of antibodies and in immunoassays.

In screening for CT genes, a Southern blot may be performed using the foregoing conditions, together with a detectably labeled probe (e.g., radioactive or chemiluminescent probes). After washing the membrane to which the DNA is finally transferred, the membrane can be placed against X-ray film or analyzed using a phosphorimager device to detect the radioactive or chemiluminescent signal. In screening for the expression of CT nucleic acids, Northern blot hybridizations using the foregoing conditions can be performed on samples taken from cancer patients or subjects suspected of having a condition characterized by abnormal cell proliferation or neoplasia. Amplification protocols such as polymerase chain reaction using primers that hybridize to the sequences presented also can be used for detection of the CT genes or expression thereof.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., testis). One can also use expression cloning utilizing the antisera described herein to identify nucleic acids that encode related antigenic proteins in humans or other species using the SEREX procedure to screen the appropriate expression libraries. (See: Sahin et al., 1995, Proc. Natl. Acad. Sci. U.S.A. 92:11810-11813).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating CT polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as antigenicity, receptor binding, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on.

In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of activity or structural relation to the nucleic acids and/or polypeptides disclosed herein. As used herein the terms: “deletion”, “addition”, and “substitution” mean deletion, addition, and substitution changes to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acids of a sequence of the invention.

According to yet another aspect of the invention, an expression vector comprising any of the isolated nucleic acid molecules of the invention, preferably operably linked to a promoter is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences.

Examples of regulatory sequences are described in Goeddel; Gene Expression Technology: Methods in Enzymology, Academic Press, San Diego, Calif. (1990). Useful regulatory expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system. It should be understood that the design of the expression vector may depend on such factors as the choice of the host cell to be transfected/transformed and/or the type of protein desired to be expressed. Moreover, the vector's copy number, the ability to control that copy number and the expression of any other protein encoded by the vector, such as antibiotic markers, should also be considered. The choice and design of an appropriate vector is within the ability and discretion of one of ordinary skill in the art.

It will also be recognized that the invention embraces the use of the CT nucleic acid molecules and genomic sequences in expression vectors, as well as to transfect host cells and cell lines, be these prokaryotic, e.g., E. coli (e.g. pBR322-derived plasmids, pEMBL-derived plasmids, pEX-derived plasmids, pBTac-derived plasmids and pUC-derived plasmids), or eukaryotic, e.g., CHO cells, COS cells (pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors, as well as vector derivatives of viruses such as the bovine papilloma virus (BPV-1), or Epstein-Barr virus (pHEBo, pREP-derived and p205), yeast expression systems, and recombinant baculovirus expression in insect cells (e.g. pVL-derived vectors: pVL1392, pVL1393 and pVL941; pAcUW1; and pBlueBac III). Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

The invention, in one aspect, also permits the construction of CT gene “knock-outs” and “knock-ins” in cells and in animals, providing materials for studying certain aspects of cancer and immune system responses to cancer.

Expression vectors containing all the necessary elements for expression are commercially available and known to those skilled in the art. Cells are genetically engineered by the introduction into the cells of heterologous DNA or RNA encoding a CT polypeptide, a mutant CT polypeptide, fragments, or variants thereof. The heterologous DNA or RNA is placed under operable control of transcriptional elements to permit the expression of the heterologous DNA in the host cell.

Preferred systems for mRNA expression in mammalian cells are those such as pcDNA3.1 and pCDM8 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is described by Warnier et al., in intradermal injection in mice for immunization against P1A (Int. J. Cancer, 67:303-310, 1996).

The invention also embraces kits termed expression kits, which allow the artisan to prepare a desired expression vector or vectors. Such expression kits include at least separate portions of each of the previously discussed coding sequences. Other components may be added, as desired, as long as the previously mentioned sequences, which are required, are included.

The invention also includes kits for amplification of a CT nucleic acid (CT1.1 (CTSP-5), CT1.11 (CTSP-6) splice variants: CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9)), including at least one pair of amplification primers which hybridize to a CT nucleic acid. The primers preferably are about 12, 13, 14, 15, 16, 17, 18, 19, 20, 21, 22, 23, 24, 25, 26, 27, 28, 29, 30, 31 or 32 nucleotides in length and are non-overlapping to prevent formation of “primer-dimers”. One of the primers will hybridize to one strand of the CT nucleic acid and the second primer will hybridize to the complementary strand of the CT nucleic acid, in an arrangement that permits amplification of the CT nucleic acid. Selection of appropriate primer pairs is standard in the art. For example, the selection can be made with assistance of a computer program designed for such a purpose, optionally followed by testing the primers for amplification specificity and efficiency.

The invention, in another aspect provides isolated polypeptides (including whole proteins and partial proteins) encoded by the foregoing CT nucleic acids CT1.1 (CTSP-5), CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9). Examples of the amino acid sequences encoded by the foregoing CT nucleic acids are set forth as any of SEQ ID NOs: 29-33, as well as CT1.19 (CTSP-7) and CT1.26 (CTSP-8). The amino acids of the invention are also intended to encompass amino acid sequences that result from the translation of the nucleic acid sequences provided herein in a different reading frame.

Such polypeptides are useful, for example, alone or as fusion proteins to generate antibodies, and as components of an immunoassay or diagnostic assay. Immunogenic CT polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Fragments of the immunogenic CT polypeptides (including immunogenic peptides) also can be synthesized chemically using well-established methods of peptide synthesis. Thus, fragments of the disclosed polypeptides are useful for eliciting an immune response. In one embodiment fragments of a polypeptide which comprises any of SEQ ID NO: 29-33 that are at least eight amino acids in length and exhibit immunogenicity are provided. In one embodiment fragments of a polypeptide which comprises CT1.19 (CTSP-7) and CT1.26 (CTSP-8) amino acid sequences that are at least eight amino acids in length and exhibit immunogenicity are provided.

Fragments of a polypeptide preferably are those fragments that retain a distinct functional capability of the polypeptide. Functional capabilities that can be retained in a fragment of a polypeptide include interaction with antibodies or MHC molecules (e.g. immunogenic fragments), interaction with other polypeptides or fragments thereof, selective binding of nucleic acids or proteins, and enzymatic activity. One important activity is the ability to provoke in a subject an immune response. As will be recognized by those skilled in the art, the size of the fragment that can be used for inducing an immune response will depend upon factors such as whether the epitope recognized by an antibody is a linear epitope or a conformational epitope or the particular MHC molecule that binds to and presents the fragment (e.g., HLA class I or II). Thus, some immunogenic fragments of CT polypeptides will consist of longer segments while others will consist of shorter segments, (e.g., about 5, 6, 7, 8, 9, 10, 11 or 12 or more amino acids long, including each integer up to the full length of the CT polypeptide). Those skilled in the art are well versed in methods for selecting immunogenic fragments of polypeptides.

The invention embraces variants of the CT polypeptides described above. As used herein, a “variant” of a CT polypeptide is a polypeptide which contains one or more modifications to the primary amino acid sequence of a CT polypeptide. Modifications which create a CT polypeptide variant can be made to a CT polypeptide 1) to reduce or eliminate an activity of a CT polypeptide; 2) to enhance a property of a CT polypeptide, such as protein stability in an expression system or the stability of protein-protein binding; 3) to provide a novel activity or property to a CT polypeptide, such as addition of an antigenic epitope or addition of a detectable moiety; or 4) to provide equivalent or better binding to a MHC molecule.

Modifications to a CT polypeptide are typically made to the nucleic acid which encodes the CT polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the CT polypeptide amino acid sequence. One of skill in the art will be familiar with methods for predicting the effect on protein conformation of a change in protein sequence, and can thus “design” a variant CT polypeptide according to known methods. One example of such a method is described by Dahiyat and Mayo in Science 278:82-87, 1997, whereby proteins can be designed de novo. The method can be applied to a known protein to vary only a portion of the polypeptide sequence. By applying the computational methods of Dahiyat and Mayo, specific variants of a CT polypeptide can be proposed and tested to determine whether the variant retains a desired conformation.

In general, variants include CT polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a CT polypeptide by eliminating proteolysis by proteases in an expression system (e.g., dibasic amino acid residues in yeast expression systems in which KEX2 protease activity is present).

Mutations of a nucleic acid which encode a CT polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant CT polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a CT gene or cDNA clone to enhance expression of the polypeptide. The activity of variants of CT polypeptides can be tested by cloning the gene encoding the variant CT polypeptide into a bacterial or mammalian expression vector, introducing the vector into an appropriate host cell, expressing the variant CT polypeptide, and testing for a functional capability of the CT polypeptides as disclosed herein. For example, the variant CT polypeptide can be tested for reaction with autologous or allogeneic sera as described in the Examples. Preparation of other variant polypeptides may favor testing of other activities, as will be known to one of ordinary skill in the art.

The skilled artisan will also realize that conservative amino acid substitutions may be made in immunogenic CT polypeptides to provide functionally equivalent variants, or homologs of the foregoing polypeptides, i.e., the variants retain the functional capabilities of the immunogenic CT polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the CT polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the CT polypeptides disclosed herein and retain the specific antibody-binding characteristics of the antigens.

Likewise, upon determining that a peptide derived from a CT polypeptide is presented by an MHC molecule and recognized by antibodies or T lymphocytes (e.g., helper T cells or CTLs), one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or T lymphocytes when bound to MHC. These variants can be tested for improved stability and are useful, inter alia, in vaccine compositions.

Conservative amino-acid substitutions in the amino acid sequence of CT polypeptides to produce functionally equivalent variants of CT polypeptides typically are made by alteration of a nucleic acid encoding a CT polypeptide. Such substitutions can be made by a variety of methods known to one of ordinary skill in the art. For example, amino acid substitutions may be made by PCR-directed mutation, site-directed mutagenesis according to the method of Kunkel (Kunkel, 1985, Proc. Nat. Acad. Sci. U.S.A. 82: 488-492), or by chemical synthesis of a gene encoding a CT polypeptide. Where amino acid substitutions are made to a small unique fragment of a CT polypeptide, such as an antigenic epitope recognized by autologous or allogeneic sera or T lymphocytes, the substitutions can be made by directly synthesizing the peptide. The activity of functionally equivalent variants of CT polypeptides can be tested by cloning the gene encoding the altered CT polypeptide into a bacterial or mammalian expression vector, introducing the vector into an appropriate host cell, expressing the altered polypeptide, and testing for a functional capability of the CT polypeptides as disclosed herein. Peptides that are chemically synthesized can be tested directly for function, e.g., for binding to antisera recognizing associated antigens.

As used herein, a “subject” is preferably a human, non-human primate, cow, horse, pig, sheep, goat, dog, cat or rodent. In all embodiments, human subjects are preferred. In some embodiments, the subject is suspected of having cancer or has been diagnosed with cancer. Cancers in which the CT nucleic acid or polypeptide are differentially expressed include, but are not limited to, cancers of the breast, colon, esophagus, glioblastoma, lung, melanoma, prostate, stomach, thyroid, and uterus.

As used herein, a biological sample includes, but is not limited to: tissue, cells, and/or body fluid (e.g., serum, blood, lymph node fluid, etc.). The fluid sample may include cells and/or fluid. The tissue and cells may be obtained from a subject or may be grown in culture (e.g., from a cell line). As used herein, a biological sample is body fluid, tissue or cells obtained from a subject using methods well-known to those of ordinary skill in the related medical arts. Typically, a biological sample may be obtained by collecting a blood sample or a biopsy sample from a subject. The biological sample can include tumor tissue or cells, normal tissue or cells, or combinations thereof.

The invention in another aspect permits the isolation of the cancer-associated antigens described herein. A variety of methodologies well-known to the skilled practitioner can be utilized to obtain isolated cancer-associated antigens. The proteins may be purified from cells which naturally produce the protein by chromatographic means or immunological recognition. Alternatively, an expression vector may be introduced into cells to cause production of the protein. In another method, mRNA transcripts may be microinjected or otherwise introduced into cells to cause production of the encoded protein. Translation of mRNA in cell-free extracts such as the reticulocyte lysate system also may be used to produce the protein. Those skilled in the art also can readily follow known methods for isolating cancer-associated antigens. These include, but are not limited to, chromatographic techniques such as immunochromatography, HPLC, size-exclusion chromatography, ion-exchange chromatography, and immune-affinity chromatography.

The invention also involves diagnosing or monitoring cancer in subjects by determining the presence of an immune response to one or more CT polypeptides of the invention (CT1.1 (CTSP-5), CT1.11 (CTSP-6), CT1.19 (CTSP-7), CT1.26 (CTSP-8), and CT1.29 (CTSP-9)). In preferred embodiments, this determination is performed by assaying a bodily fluid obtained from the subject, preferably serum, blood, or lymph node fluid for the presence of antibodies against the CT polypeptides described herein. This determination may also be performed by assaying a tissue or cells from the subject for the presence of one or more CT polypeptides (or nucleic acid molecules that encode these polypeptides) described herein. In another embodiment, the presence of antibodies against at least one additional cancer-associated antigen or cancer-testis antigen is determined for diagnosis of cancer. This determination may also be performed by assaying a tissue or cells from the subject for the presence of the CT polypeptides described herein.

Measurement of the expression of CT polypeptides or nucleic acid molecules, or the immune response against one of the CT polypeptides, over time by sequential determinations permits monitoring of the disease and/or the effects of a course of treatment. For example, a sample, such as serum, blood, or lymph node fluid, may be obtained from a subject, tested for expression of CT molecules or an immune response to one of the CT polypeptides, and at a second, subsequent time, another sample, may be obtained from the subject and similarly tested. The results of the first and second (or subsequent) tests can be compared as a measure of the onset, regression or progression of cancer, or, if cancer treatment was undertaken during the interval between obtaining the samples, the effectiveness of the treatment may be evaluated by comparing the results of the two tests. In preferred embodiments of immune response testing, the CT polypeptides are bound to a substrate and/or the immune response to the CT polypeptides is determined with ELISA. Other methods will be apparent to one of skill in the art.

Diagnostic methods of the invention also involve determining the aberrant expression of one or more of the CT polypeptides (CT1.1 (CTSP-5), CT1.11 (CTSP-6), CT1.19 (CTSP-7), CT1.26 (CTSP-8), and CT1.29 (CTSP-9)) described herein or the nucleic acid molecules that encode them. Such determinations can be carried out via any standard nucleic acid assay, including the polymerase chain reaction or assaying with hybridization probes, which may be labeled, or by assaying biological samples with binding partners (e.g., antibodies) for CT polypeptides.

The diagnostic methods of the invention can be used to detect the presence of a disorder associated with aberrant expression of a CT molecule (e.g., onset of the disorder), as well as to assess the progression and/or regression of the disorder such as in response to treatment (e.g., chemotherapy, radiation). According to this aspect of the invention, the method for diagnosing a disorder characterized by aberrant expression of a CT molecule involve: detecting expression of a CT molecule in a first biological sample obtained from a subject, wherein differential expression of the CT molecule compared to a control sample indicates that the subject has a disorder characterized by aberrant expression of a CT molecule, such as cancer.

As described herein, CT molecule expression is restricted to testis tissue (CT1.1 (CTSP-5), CT1.19 (CTSP-7), CT1.26 (CTSP-8), and CT1.29 (CTSP-9)). Certain CT molecules may have restricted expression in additional normal tissues, e.g. placenta for CT1.11 (CTSP-6). Therefore, in all of the diagnostic methods described herein, the biological sample preferably does not contain testis cells or testis tissue in order to avoid false-positive results. For CT1.11 (CTSP-6), the sample preferably does not contain testis and placental cells or tissue.

As used herein, “aberrant expression” of a CT molecule is intended to include any expression that is statistically significant different from the expected (e.g., normal or baseline) amount of expression. For example, expression of a CT molecule (i.e., CT polypeptides or the nucleic acid molecules that encode them) in a tissue that is not expected to express the CT molecule would be included in the definition of “aberrant expression”. Likewise, expression of the CT molecule that is determined to be expressed at a significantly higher or lower level than expected is also included. Therefore, a determination of the level of expression of one or more of the CT polypeptides and/or the nucleic acids that encode them is diagnostic of cancer if the level of expression is above a baseline level determined for that tissue type. The baseline level of expression can be determined using standard methods known to those of skill in the art. Such methods include, for example, assaying a number of histologically normal tissue samples (preferably not testis) from subjects that are clinically normal (i.e. do not have clinical signs of cancer in that tissue type) and determining the mean level of expression for the samples.

The level of expression of the nucleic acid molecules of the invention or the polypeptides they encode can indicate cancer in the tissue when the level of expression is significantly more in the tissue than in a control sample. In some embodiments, a level of expression in the tissues that is at least about 5%,10%, 20%, 30%, 40%, 50%, 60%, 70%, 80%, 90%, 100%, 150%, 200%, 250%, 300%, 400%, or 500% more than the level of expression in the control tissue indicates cancer in the tissue.

As used herein the term “control” means predetermined values, and also means samples of materials tested in parallel with the experimental materials. Examples include samples from control populations, biopsy samples taken from tissue adjacent to a biopsy sample suspected of being cancerous and control samples generated through manufacture to be tested in parallel with the experimental samples.

As used herein the term “control” includes positive and negative controls which may be a predetermined value that can take a variety of forms. The control(s) can be a single cut-off value, such as a median or mean, or can be established based upon comparative groups, such as in groups having normal amounts of CT molecules of the invention and groups having abnormal amounts of CT molecules of the invention. Another example of a comparative group is a group having a particular disease, condition and/or symptoms and a group without the disease, condition and/or symptoms. Another comparative group is a group with a family history of a particular disease and a group without such a family history of the particular disease. The predetermined control value can be arranged, for example, where a tested population is divided equally (or unequally) into groups, such as a low-risk group, a medium-risk group and a high-risk group or into quadrants or quintiles, the lowest quadrant or quintile being individuals with the lowest risk or lowest expression levels of a CT molecule of the invention that is up-regulated in cancer and the highest quadrant or quintile being individuals with the highest risk or highest expression levels of a CT molecule of the invention that is up-regulated in cancer.

The predetermined value of a control will depend upon the particular population selected. For example, an apparently healthy population will have a different “normal” CT molecule expression level range than will a population which is known to have a condition characterized by aberrant expression of the CT molecule. Accordingly, the predetermined value selected may take into account the category in which an individual falls. Appropriate ranges and categories can be selected with no more than routine experimentation by those of ordinary skill in the art. Typically the control will be based on apparently healthy individuals in an appropriate age bracket. As used herein, the term “increased expression” means a higher level of expression relative to a selected control.

The invention involves in some aspects diagnosing or monitoring cancer by determining the level of expression of one or more CT nucleic acid molecules and/or determining the level of expression of one or more CT polypeptides they encode. In some important embodiments, this determination is performed by assaying a tissue sample from a subject for the level of expression of one or more CT nucleic acid molecules or for the level of expression of one or more CT polypeptides encoded by the nucleic acid molecules of the invention (CT1.1 (CTSP-5), CT1.11 (CTSP-6), CT1.19 (CTSP-7), CT1.26 (CTSP-8), and CT1.29 (CTSP-9)).

The expression of the molecules of the invention may be determined using routine methods known to those of ordinary skill in the art. These methods include, but are not limited to: direct RNA amplification, reverse transcription of RNA to cDNA, real-time RT-PCR, amplification of cDNA, hybridization, and immunologically based assay methods, which include, but are not limited to immunohistochemistry, antibody sandwich capture assay, ELISA, and enzyme-linked immunospot assay (EliSpot assay). For example, the determination of the presence of level of nucleic acid molecules of the invention in a subject or tissue can be carried out via any standard nucleic acid determination assay, including the polymerase chain reaction, or assaying with labeled hybridization probes. Such hybridization methods include, but are not limited to microarray techniques.

These methods of determining the presence and/or level of the molecules of the invention in cells and tissues may include use of labels to monitor the presence of the molecules of the invention. Such labels may include, but are not limited to, radiolabels or chemiluminescent labels, which may be utilized to determine whether a molecule of the invention is expressed in a cell or tissue, and to determine the level of expression in the cell or tissue. For example, a fluorescently labeled or radiolabeled antibody that selectively binds to a polypeptide of the invention may be contacted with a tissue or cell to visualize the polypeptide in vitro or in vivo. These and other in vitro and in vivo imaging methods for determining the presence of the nucleic acid and polypeptide molecules of the invention are well known to those of ordinary skill in the art.

The invention, therefore, also involves the use of agents such as polypeptides that bind to CT polypeptides. Such agents can be used in methods of the invention including the diagnosis and/or treatment of cancer. Such binding agents can be used, for example, in screening assays to detect the presence or absence of CT polypeptides and can be used in quantitative binding assays to determine levels of expression in biological samples and cells. Such agents also may be used to inhibit the native activity of the CT polypeptides, for example, by binding to such polypeptides.

According to this aspect, the binding polypeptides bind to an isolated nucleic acid or protein of the invention, including unique fragments thereof. Preferably, the binding polypeptides bind to a CT polypeptide, or a unique fragment thereof.

In preferred embodiments, the binding polypeptide is an antibody or antibody fragment, more preferably, an Fab or F(ab)₂ fragment of an antibody. Typically, the fragment includes a CDR3 region that is selective for the CT polypeptide. Any of the various types of antibodies can be used for this purpose, including polyclonal antibodies, monoclonal antibodies, humanized antibodies, and chimeric antibodies.

Thus, the invention provides agents which bind to CT polypeptides encoded by CT nucleic acid molecules of the invention, and in certain embodiments preferably to unique fragments of the CT polypeptides. Such binding partners can be used in screening assays to detect the presence or absence of a CT polypeptide and in purification protocols to isolate such CT polypeptides. Likewise, such binding partners can be used to selectively target drugs, toxins or other molecules (including detectable diagnostic molecules) to cells which express CT polypeptides. In this manner, for example, cells present in solid or non-solid tumors which express CT polypeptides can be treated with cytotoxic compounds that are selective for the CT molecules (nucleic acids and/or antigens). Such binding agents also can be used to inhibit the native activity of the CT polypeptide, for example, to further characterize the functions of these molecules.

The antibodies of the present invention thus are prepared by any of a variety of methods, including administering a protein, fragments of a protein, cells expressing the protein or fragments thereof and the like to an animal to induce polyclonal antibodies. The present invention also provides methods of producing monoclonal antibodies to the CT molecules of the invention described herein. The production of monoclonal antibodies is performed according to techniques well known in the art. As detailed herein, such antibodies may be used for example to identify tissues expressing protein or to purify protein. Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

Significantly, as is well-known in the art, only a small portion of an antibody molecule, the paratope, is involved in the binding of the antibody to its epitope (see, in general, Clark, W. R., 1986, The Experimental Foundations of Modern Immunology, Wiley & Sons, Inc., New York; Roitt, I., 1991, Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)2, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies, domain antibodies and heavy chain antibodies (Ablynx N V, Ghent, Belgium). Thus, the invention involves polypeptides of numerous sizes and types that bind specifically to CT polypeptides (CT1.1 (CTSP-5), CT1.11 (CTSP-6), CT1.19 (CTSP-7), CT1.26 (CTSP-8), and CT1.29 (CTSP-9)). These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

The CT polypeptides of the invention can be used to screen peptide libraries, including phage display libraries, to identify and select peptide binding partners of the CT molecules of the invention. Such molecules can be used, as described, for screening assays, for diagnostic assays, for purification protocols or for targeting drugs, toxins and/or labeling agents (e.g., radioisotopes, fluorescent molecules, etc.) to cells which express CT molecules such as cancer cells which have aberrant CT expression.

Phage display can be particularly effective in identifying binding peptides useful according to the invention. Briefly, one prepares a phage library (using e.g., m13, fd, or lambda phage), displaying inserts from 4 to about 80 amino acid residues using conventional procedures. The inserts may represent, for example, a completely degenerate or biased array. One then can select phage-bearing inserts which bind to the CT polypeptide. This process can be repeated through several cycles of reselection of phage that bind to the CT polypeptide. Repeated rounds lead to enrichment of phage bearing particular sequences. DNA sequence analysis can be conducted to identify the sequences of the expressed polypeptides. The minimal linear portion of the sequence that binds to the CT polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the CT polypeptides.

As detailed herein, the foregoing antibodies and other binding molecules may be used to identify tissues with normal or aberrant expression of a CT polypeptide. Antibodies also may be coupled to specific diagnostic labeling agents for imaging of cells and tissues with normal or aberrant CT polypeptide expression or to therapeutically useful agents according to standard coupling procedures. As used herein, “therapeutically useful agents” include any therapeutic molecule which desirably is targeted selectively to a cell or tissue selectively with an aberrant CT expression.

Diagnostic agents for in vivo use include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99, iodine-131 and indium-111, and nuclides for nuclear magnetic resonance such as fluorine and gadolinium. Other diagnostic agents useful in the invention will be apparent to one of ordinary skill in the art.

The antibodies of the present invention can also be used to therapeutically target CT polypeptides. In one embodiment, antibodies can be used to target CT antigens expressed on the cell surface, such as CT peptides presented by MHC molecules. This can be accomplished, for example, by raising antibodies that recognize the complex of CT peptides and MHC molecules.

These antibodies can be linked not only to a detectable marker but also an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents that may be conjugated to the antibodies of the present invention include dolastatins (U.S. Pat. Nos. 6,034,065 and 6,239,104) and derivatives thereof. Of particular interest is dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., 1998, Anticancer Drug Des. 13(4):243-277; Woyke, T. et al., 2001, Antimicrob. Agents Chemother. 45(12):3580-3584), and aurastatin E and the like. Toxins that are less preferred in the compositions and methods of the invention include poisonous lectins, plant toxins such as ricin, abrin, modeccin, botulinum and diphtheria toxins. Of course, combinations of the various toxins could also be coupled to one antibody molecule thereby accommodating variable cytotoxicity. Other chemotherapeutic agents are known to those skilled in the art.

Agents that act on the tumor vasculature can include tubulin-binding agents such as combrestatin A4 (Griggs et al., 2001, Lancet Oncol. 2:82), angiostatin and endostatin (reviewed in Rosen, 2000, Oncologist 5:20, incorporated by reference herein) and interferon inducible protein 10 (U.S. Pat. No. 5,994,292). A number of antiangiogenic agents currently in clinical trials are also contemplated. Agents currently in clinical trials include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. Additional antiangiogenic agents are described by Kerbel, 2001, J. Clin. Oncol. 19(18s):45s-51s, which is incorporated by reference herein. Immunomodulators suitable for conjugation to the antibodies include α-interferon, γ-interferon, and tumor necrosis factor alpha (TNFα).

The coupling of one or more toxin molecules to the antibody is envisioned to include many chemical mechanisms, for instance covalent binding, affinity binding, intercalation, coordinate binding, and complexation. The toxic compounds used to prepare the immunotoxins are attached to the antibodies or antigen-binding fragments thereof by standard protocols known in the art.

In other aspects of the invention, the CT molecules and the antibodies and other binding molecules, as described herein, can be used for the treatment of disorders. When “disorder” is used herein, it refers to any pathological condition wherein the CT polypeptides are aberrantly expressed. An example of such a disorder is cancer, including but not limited to, breast cancer, lung cancer, head and neck cancer, colon cancer, prostate cancer, esophageal cancer, brain cancers such as glioblastoma, melanoma, stomach cancer, thyroid cancer, uterine cancer, ovarian cancer, colorectal cancer, renal cancer, sarcoma, rhabdomyosarcoma, leukemia, lymphoma, myeloma, gastric cancer, glioma, bladder cancer, and hepatoma.

Conventional treatment for cancer may include, but is not limited to: surgical intervention, chemotherapy, radiotherapy, and adjuvant systemic therapies. In one aspect of the invention, treatment may include administering binding polypeptides such as antibodies that specifically bind to a CT polypeptide (CT1.1 (CTSP-5), CT1.11 (CTSP-6), CT1.19 (CTSP-7), CT1.26 (CTSP-8), and CT1.29 (CTSP-9)). These binding polypeptides can be optionally linked to one or more detectable markers, antitumor agents or immunomodulators as described above.

Cancer treatment, in another aspect of the invention may include administering antisense molecules or RNAi molecules to reduce expression level and/or function level of CT polypeptides of the invention in the subject in cancers where a CT molecule is up-regulated.

Polynucleotides that comprise an antisense sequence act through an antisense mechanism for inhibiting expression of the CT gene (CT1.1 (CTSP-5), CT1.11 (CTSP-6), CT1.19 (CTSP-7), CT1.26 (CTSP-8), and CT1.29 (CTSP-9)). Antisense technologies have been widely utilized to regulate gene expression (Buskirk et al., Chem Biol 11, 1157-63 (2004); and Weiss et al., Cell Mol Life Sci 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hinderance, altering splicing, or inducing cleavage or other enzymatic inactivation of the transcript. The binding may be by conventional base pair complementarity, or, for example, in the case of binding to DNA duplexes, through specific interactions in the major groove of the double helix. In general, “antisense” technology refers to the range of techniques generally employed in the art, and includes any therapy that relies on specific binding to nucleic acid sequences.

A polynucleotide that comprises an antisense sequence of the present invention can be delivered, for example, as a component of an expression plasmid which, when transcribed in the cell, produces a nucleic acid sequence that is complementary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention may be modified so that they are resistant to endogenous nucleases, e.g. exonucleases and/or endonucleases, and are therefore stable in vivo. Examples of nucleic acid molecules for use in polynucleotides of the invention are phosphoramidate, phosphorothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der Krol et al. (1988) Biotechniques 6:958-976; and Stein et al. (1988) Cancer Res 48:2659-2668. Antisense approaches involve the design of polynucleotides (either DNA or RNA) that are complementary to a target nucleic acid encoding a CT gene (CT1.1 (CTSP-5), CT1.11 (CTSP-6), CT1.19 (CTSP-7), CT1.26 (CTSP-8), and CT1.29 (CTSP-9)). The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have recently been shown to be effective at inhibiting translation of mRNAs as well (Wagner, R. 1994. Nature 372:333). Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad Sci. USA 86:6553-6556; Lemaitre et al., 1987, Proc Natl Acad Sci. USA 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

Polynucleotides of the invention may also comprise at least one modified sugar moiety selected from the group including but not limited to arabinose, 2-fluoroarabinose, xylulose, and hexose.

A polynucleotide of the invention can also contain a neutral peptide-like backbone. Such molecules are termed peptide nucleic acid (PNA)-oligomers and are described, e.g., in Perry-O'Keefe et al. (1996) Proc. Natl. Acad. Sci. USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc. Natl. Acad. Sci. USA 85:7448-7451 (1988)).

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

Another approach utilizes a recombinant DNA construct in which the antisense polynucleotide is placed under the control of a strong pol III or pol II promoter. The use of such a construct to transfect target cells in the patient will result in the transcription of sufficient amounts of antisense polynucleotides that will form complementary base pairs with the CT gene or mRNA and thereby attenuate the activity of the CT protein. For example, a vector can be introduced in vivo such that it is taken up by a cell and directs the transcription of an antisense polynucleotide that targets a CT gene or mRNA. Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionine gene (Brinster et al, Nature 296:3942 (1982)), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

A further aspect of the invention includes inhibitor molecules that are short interfering nucleic acids (siNA), which include, short interfering RNA (siRNA), double-stranded RNA (dsRNA), micro-RNA (miRNA), and short hairpin RNA (shRNA) molecules, and that are used to inhibit the expression of target genes. The siNAs of the present invention, for example siRNAs, typically regulate gene expression via target RNA transcript cleavage/degradation or translational repression of the target messenger RNA (mRNA). In one embodiment siRNAs are exogenously delivered to a cell. In a specific embodiment siRNA molecules are generated that specifically target (CT1.1 (CTSP-5), CT1.11 (CTSP-6) splice variants: CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9)).

A short interfering nucleic acid (siNA) of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of inhibiting gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. For example, in some cases, siRNAs are modified to alter potency, target affinity, the safety profile and/or the stability to render them resistant or partially resistant to intracellular degradation. Modifications, such as phosphorothioates, for example, can be made to siRNAs to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006). siRNAs with amide-linked oligoribonucleosides have been generated that are more resistant to S1 nuclease degradation (Iwase R et al. 2006 Nucleic Acids Symp Ser 50: 175-176). In addition, modification of siRNA at the 2′-sugar position and phosphodiester linkage confers improved serum stability without loss of efficacy (Choung et al., Biochem. Biophys. Res. Commun. 342(3):919-26, 2006). In one study, 2′-deoxy-2′-fluoro-beta-D-arabinonucleic acid (FANA)-containing antisense oligonucleotides compared favourably to phosphorothioate oligonucleotides, 2′-O-methyl-RNA/DNA chimeric oligonucleotides and siRNAs in terms of suppression potency and resistance to degradation (Ferrari N et a. 2006 Ann N Y Acad Sci 1082: 91-102).

In some embodiments an siNA is an shRNA molecule encoded by and expressed from a genomically integrated transgene or a plasmid-based expression vector. Thus, in some embodiments a molecule capable of inhibiting gene expression is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

One embodiment herein contemplates the use of gene therapy to deliver one or more expression vectors, for example viral-based gene therapy, encoding one or more small interfering nucleic acids, capable of inhibiting expression of CT^{S}. As used herein, gene therapy is a therapy focused on treating genetic diseases, such as cancer, by the delivery of one or more expression vectors encoding therapeutic gene products, including polypeptides or RNA molecules, to diseased cells. Methods for construction and delivery of expression vectors will be known to one of ordinary skill in the art. CT polypeptides as described herein, can also be used in one aspect of the invention to induce or enhance an immune response. For example, the CT polypeptides of the invention may be used to stimulate lymphocytes, eliciting a B cell response and/or T cell response. Non-limiting examples of T cell responses include CD4+ T cell responses and CD8+ cell responses. Some therapeutic approaches based upon the disclosure are premised on a response by a subject's immune system, leading to lysis of antigen presenting cells, such as cancer cells which present one or more CT polypeptides of the invention. One such approach is the administration of autologous CTLs specific to a CT polypeptide/MHC complex to a subject with abnormal cells of the phenotype at issue. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has been described (Altman et al., 1996, Science 274:94-96; Dunbar et al., 1998, Curr. Biol. 8:413-416), in which fluorogenic tetramers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g., phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

To detail a therapeutic methodology, referred to as adoptive transfer (Greenberg, 1986, J. Immunol. 136(5): 1917; Riddel et al., 1992, Science 257: 238; Lynch et al., 1991, Eur. J. Immunol. 21: 1403-1410; Kast et al., 1989, Cell 59: 603-614), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/cancer associated antigen complex. This can be determined very easily, as the art is very familiar with methods for identifying cells which present a particular HLA molecule, as well as how to identify cells expressing DNA of the pertinent sequences, in this case a CT polypeptide sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a CT polypeptide is being presented, and the subject is an appropriate candidate for the therapeutic approaches set forth supra.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e., the antigenic peptide and the presenting MHC molecule). Chen et al. (Proc. Natl. Acad. Sci. U.S.A. 88: 110-114, 1991) exemplifies this approach, showing the use of transfected cells expressing HPV E7 peptides in a therapeutic regime. Various cell types may be used. Similarly, vectors carrying one or both of the genes of interest may be used. Viral or bacterial vectors are especially preferred. For example, nucleic acids which encode a CT polypeptide may be operably linked to promoter and enhancer sequences which direct expression of the CT polypeptide in certain tissues or cell types. The nucleic acid may be incorporated into an expression vector.

Expression vectors may be unmodified extrachromosomal nucleic acids, plasmids or viral genomes constructed or modified to enable insertion of exogenous nucleic acids, such as those encoding CT polypeptide, as described elsewhere herein. Nucleic acids encoding a CT polypeptide also may be inserted into a retroviral genome, thereby facilitating integration of the nucleic acid into the genome of the target tissue or cell type. In these systems, the gene of interest is carried by a microorganism, e.g., a Vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the CT polypeptide or a stimulatory fragment thereof with an adjuvant to facilitate incorporation into antigen presenting cells in vivo. The CT polypeptide is processed to yield the peptide partner of the MHC molecule while a CT fragment may be presented without the need for further processing. Generally, subjects can receive an intradermal injection of an effective amount of the CT polypeptide. Initial doses can be followed by booster doses, following immunization protocols standard in the art. Preferred CT polypeptides include those found to react with allogeneic cancer antisera, shown in the examples below.

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models, can be used for testing of immunization against cancer using a CT molecule. For example, human cancer cells can be introduced into a mouse to create a tumor, and one or more CT polypeptides (CT1.1 (CTSP-5), CT1.11 (CTSP-6), CT1.19 (CTSP-7), CT1.26 (CTSP-8), and CT1.29 (CTSP-9)) or fragments thereof can be delivered, optionally combined with one or more adjuvants and/or cytokines to boost the immune response. The effect on the cancer cells (e.g., reduction of tumor size) can be assessed as a measure of the effectiveness of the CT immunization. Testing of the foregoing animal model using other methods for immunization include the administration of one or more CT nucleic acids or fragments derived therefrom.

Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immunization compositions, one or more CT polypeptides or immunogenic fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); QS-7, QS-17, QS-18, and QS-L1 (So et al., 1997, Mol. Cells 7:178-186); incomplete Freund's adjuvant; complete Freund's adjuvant; montanide; alum; CpG oligonucleotides (see e.g., Krieg et al., 1995, Nature 374:546-9); and various water-in-oil emulsions prepared from biodegradable oils such as squalene and/or tocopherol. Preferably, the antigens are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g., Goding, Monoclonal Antibodies: Principles and Practice, 2nd Ed., 1986). Methods for the preparation of mixtures or emulsions of polypeptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation to an antigen/MHC/TCR stimulated T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P. et al., 1998, Proc. Natl. Acad. Sci. U.S.A. 95 (11):6284-6289).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., 1995, J. Immunol, 154:5637-5648). Tumor cell transfection with B7 has been discussed in relation to in vitro CTL expansion for adoptive transfer immunotherapy by Wang et al., (J. Immunol., 19:1-8, 1986). Other delivery mechanisms for the B7 molecule would include nucleic acid (naked DNA) immunization (Kim J., et al., 1997, Nat. Biotechnol., 15(7):641-646) and recombinant viruses such as adeno and pox (Wendtner et al., 1997, Gene Ther., 4(7):726-735). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., 1999, Nature 397:263-266).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., 1997, J. Immunol. 158:637-642; Fenton et al., 1998, J. Immunother., 21(2):95-108).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., 1998, J. Immunother., 21(2):95-108). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., 1998, Nature 393:474; Bennett et al., 1998, Nature 393:478; Schoenberger et al., 1998, Nature 393:480). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor antigens which are normally encountered outside of an inflammatory context or are presented by non-professional APCs (tumor cells). In these situations Th help and B7 costimulation signals are not provided.

The invention contemplates delivery of nucleic acids, polypeptides or fragments thereof for vaccination. Delivery of polypeptides and fragments thereof can be accomplished according to standard vaccination protocols which are well known in the art. In another embodiment, the delivery of nucleic acid is accomplished by ex vivo methods, i.e. by removing a cell from a subject, genetically engineering the cell to express or include a CT polypeptide, and reintroducing the engineered cell into the subject. One example of such a procedure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

A virus vector for delivering a nucleic acid encoding a CT polypeptide is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., 1996, Virology 219:220-227; Eloit et al., 1997, J. Virol. 7:5375-5381; Chengalvala et al., 1997, Vaccine 15:335-339), a modified retrovirus (Townsend et al., 1997, J. Virol. 71:3365-3374), a nonreplicating retrovirus (Irwin et al., 1994, J. Virol. 68:5036-5044), a replication defective Semliki Forest virus (Zhao et al., 1995, Proc. Natl. Acad. Sci. U.S.A. 92:3009-3013), canarypox virus and highly attenuated vaccinia virus derivative (Paoletti, 1996, Proc. Natl. Acad. Sci. U.S.A. 93:11349-11353), non-replicative vaccinia virus (Moss, 1996, Proc. Natl. Acad. Sci. U.S.A. 93:11341-11348), replicative vaccinia virus (Moss, 1994, Dev. Biol. Stand. 82:55-63), Venzuelan equine encephalitis virus (Davis et al., 1996, J. Virol. 70:3781-3787), Sindbis virus (Pugachev et al., 1995, Virology 212:587-594), and Ty virus-like particle (Allsopp et al., 1996, Eur. J. Immunol. 26:1951-1959). A preferred virus vector is an adenovirus.

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a CT polypeptide, alone or as a complex with a MHC molecule. Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

According to a further aspect of the invention, compositions containing the nucleic acid molecules, proteins, and binding polypeptides of the invention are provided. The compositions contain any of the foregoing therapeutic agents in a carrier, optionally a pharmaceutically acceptable carrier. Thus, in a related aspect, the invention provides a method for forming a medicament that involves placing a therapeutically effective amount of the therapeutic agent in the pharmaceutically acceptable carrier to form one or more doses. The effectiveness of treatment or prevention methods of the invention can be determined using standard diagnostic methods described herein.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral, intravenous, intratumoral, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without undue experimentation. When using antisense preparations of the invention, slow intravenous administration is preferred.

The compositions of the invention are administered in effective amounts. An “effective amount” is that amount of a CT polypeptide composition (CT1.1 (CTSP-5), CT1.11 (CTSP-6), CT1.19 (CTSP-7), CT1.26 (CTSP-8), and CT1.29 (CTSP-9)) that alone, or together with further doses, produces the desired response, e.g. increases an immune response to the CT polypeptide. In the case of treating a particular disease or condition characterized by expression of one or more CT polypeptides, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of CT polypeptide or nucleic acid encoding CT polypeptide for producing the desired response in a unit of weight or volume suitable for administration to a patient. The response can, for example, be measured by determining the immune response following administration of the CT polypeptide composition via a reporter system by measuring downstream effects such as gene expression, or by measuring the physiological effects of the CT polypeptide composition, such as regression of a tumor or decrease of disease symptoms. Other assays will be known to one of ordinary skill in the art and can be employed for measuring the level of the response.

The doses of CT polypeptide compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of CT polypeptide are formulated and administered in doses between 1 ng and 1 mg, and preferably between 10 ng and 100 μg, according to any standard procedure in the art. Where nucleic acids encoding CT polypeptides or variants thereof are employed, doses of between 1 ng and 0.1 mg generally will be formulated and administered according to standard procedures. Other protocols for the administration of CT polypeptide compositions will be known to one of ordinary skill in the art, in which the dose amount, schedule of injections, sites of injections, mode of administration (e.g., intra-tumoral) and the like vary from the foregoing. Administration of CT polypeptide compositions to mammals other than humans, e.g. for testing purposes or veterinary therapeutic purposes, is carried out under substantially the same conditions as described above.

Where CT polypeptides are used for vaccination, modes of administration which effectively deliver the CT polypeptide and adjuvant, such that an immune response to the polypeptide is increased, can be used. For administration of a CT polypeptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. Additional materials may be included in any or all kits of the invention, and such materials may include, but are not limited to buffers, water, enzymes, tubes, control molecules, etc. The kit may also include instructions for the use of the one or more pharmaceutical compounds or agents of the invention for the treatment of cancer.

The invention includes kits for assaying the presence of CT polypeptides and/or antibodies that specifically bind to CT polypeptides (CT1.1 (CTSP-5), CT1.11 (CTSP-6), CT1.19 (CTSP-7), CT1.26 (CTSP-8), and CT1.29 (CTSP-9)). An example of such a kit may include the above-mentioned polypeptides bound to a substrate, for example a dipstick, which is dipped into a blood or body fluid sample of a subject. The surface of the substrate may then be processed using procedures well known to those of skill in the art, to assess whether specific binding occurred between the polypeptides and agents (e.g., antibodies) in the subject's sample. For example, procedures may include, but are not limited to, contact with a secondary antibody, or other method that indicates the presence of specific binding.

Another example of a kit may include an antibody or antigen-binding fragment thereof, that binds specifically to a CT polypeptide. The antibody, or antigen-binding fragment thereof, may be applied to a tissue or cell sample from a patient with cancer and the sample then processed to assess whether specific binding occurs between the antibody and an antigen or other component of the sample. In addition, the antibody, or antigen-binding fragment thereof, may be applied to a body fluid sample, such as serum, from a subject, either suspected of having cancer, diagnosed with cancer, or believed to be free of cancer. As will be understood by one of skill in the art, such binding assays may also be performed with a sample or object contacted with an antibody and/or CT polypeptide that is in solution, for example in a 96-well plate, or applied directly to a solid support (i.e., an object's surface).

Another example of a kit of the invention is a kit that provides components necessary to determine the level of expression of one or more CT nucleic acid molecules of the invention. Such components may include primers useful for amplification of one or more CT nucleic acid molecules and/or other chemicals for PCR amplification.

Another example of a kit of the invention is a kit that provides components necessary to determine the level of expression of one or more CT nucleic acid molecules of the invention using a method of hybridization.

The foregoing kits can include instructions or other printed material on how to use the various components of the kits for diagnostic purposes. Kits may also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber.

The invention further includes nucleic acid or protein microarrays (including antibody arrays) for the analysis of expression of CT polypeptides or nucleic acids encoding such antigens. In this aspect of the invention, standard techniques of microarray technology are utilized to assess expression of the CT polypeptides and/or identify biological constituents that bind such antigens. The constituents of biological samples include antibodies, lymphocytes (particularly T lymphocytes), and the like. Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate. Nucleic acid probes preferably are linked using UV irradiation or heat.

Protein microarray technology, which is also known by other names including protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

Targets are peptides or proteins and may be natural or synthetic. The tissue may be obtained from a subject or may be grown in culture (e.g., from a cell line).

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

Nucleic acid arrays, particularly arrays that bind CT nucleic acid sequences (CT1.1 (CTSP-5), CT1.11 (CTSP-6) splice variants: CT1.11a (CTSP-6.1), CT1.11b (CTSP-6.2), CT1.11c (CTSP-6.3), CT1.11d (CTSP-6.4), CT1.19 (CTSP-7), CT1.26 (CTSP-8), and CT1.29 (CTSP-9)), also can be used for diagnostic applications, such as for identifying subjects that have a condition characterized by aberrant CT molecule expression, e.g., cancer. Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

According to the invention, probes are selected from the group of nucleic acids including, but not limited to: DNA, genomic DNA, cDNA, and oligonucleotides; and may be natural or synthetic. Oligonucleotide probes preferably are 15 to 40-mer oligonucleotides and DNA/cDNA probes preferably are 200 to 5000 bases in length, although other lengths may be used. Appropriate probe length may be determined by one of ordinary skill in the art by following art-known procedures. In one embodiment, preferred probes are sets of one or more of the CT nucleic acid molecules as described herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or oligonucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, nucleic acid probes are synthesized directly on the substrate in a predetermined grid pattern using methods such as light-directed chemical synthesis, photochemical deprotection, or delivery of nucleotide precursors to the substrate and subsequent probe production.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g., from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Novel Identified Cancer Testis Antigens and Their Frequency of mRNA Expression in Tumors

### Computational Strategy

### Transcriptome Database (Map4)

The database used in this work contains data obtained from alignments of cDNA sequences (mRNAs and ESTs) to the human genome sequence. All human cDNAs available in dbEST (July 2002) and mRNA sequences from known human genes from UniGene release 153 were aligned to the masked human genome sequence [build 29, obtained from the National Center for Biotechnology Information (NCBI)] by using pp-Blast, an implementation of MEGABLAST for a parallel cluster. The parameters used in MEGABLAST were: -f T -J F -F F -W 24. The MEGABLAST output was parsed and a MySQL database was loaded with the mapping information. Spurious hits were excluded from the mapping database by using an additional set of alignment criteria. These included a minimum degree of identity for a cDNA/genome alignment set to 93% over at least 45% of the total EST length or 55% of the total length of the full-insert sequence. Clustering of cDNA sequences was based on their genomic coordinates as described by Sabake et al. (2003). Briefly, if two sequences shared at least partially the same gene structure they were joined into the same cluster. If no exon/intron boundary was defined, a sequence had to have at least a 100-bp overlap with another sequence at the genome level to be added to the respective cluster. By querying the transcriptome database, it was possible to get the coordinates of each EST alignment and also the related information of each sequence, such as project and tissue source of the sequences. The information about the tissue origin of each expressed sequence was obtained from dbEST specifically in tissue and organ descriptions of each cDNA library. Libraries were classified as derived from tumor tissues if the words tumor, cancer, leukemia or the suffix “oma” were present in the description, such as “adenocarcinoma”, “glioblastoma”.

### Map4 Best Hits Database

In order to eliminate the presence of multiples alignments of expressed sequences in the human genome, the Map4_best hits database (Map4_bh) was created. Sequences mapping to more than one location on the genome were given a score for alignment quality. A higher score was associated with a higher identity over a longer alignment. Only the sequences with the highest scores were kept.

### Cluster Selection and Manual Inspection

By querying the Map4_bh database, we were able to select clusters composed of spliced ESTs derived from testis and/or tumoral cDNA libraries. The pipeline used in this work was developed by using PERL programming languages on a Linux-based server running the MySQL database management system. Clusters selected by the pipeline were manually inspected to confirm the splicing structure by checking the presence of conserved acceptor and donor splicing sites (GT/AG) and to exclude clusters that correspond to known CT antigens. Manual inspection was carried out by using the BLAT search tool provided by University of California, Santa Cruz (UCSC).

### Expression Analysis

### Samples

Total RNA derived from 21 normal human tissues (testis, lung, prostate, small intestine, breast, brain, cerebellum, heart, uterus, trachea, placenta, colon, fetal brain, fetal liver, thymus, salivary gland, spinal cord, stomach, kidney, spleen, and skeletal muscle) was purchased from Clontech (Mountain View, Calif.).

Human tumor cell lines [A172 and T98G (glioblastoma); FaDu (squamous cell carcinoma); SW480 (colorectal adenocarcinoma); Skmel-28 and A2058 (malignant melanoma); DU145 and PC3 (prostate carcinoma); HeLa and CasKi (cervix adenocarcinoma); MCF-7 and MDA-MB-436 (breast ductal carcinoma); HL60 (lymphocytes); H1155 and H358 (lung carcinoma); SCABER (urinary bladder carcinoma); SAOS-2 (osteosarcoma)] were obtained from American Type Culture Collection (Manassas, Va.) and a total of 160 tumor samples derived from 9 different types of tumors (colon, stomach, glioblastoma, breast, melanoma, prostate, lung, uterus, thyroid) were collected from patients treated at Hospital A. C. Camargo. All samples were collected after explicit informed consent and with local ethical committee approval.

RNA Extraction, cDNA Synthesis and RT-PCR

Total RNA was extracted by CsCl-guanidine thiocyanate method and RNA samples were checked for integrity by agarose gel electrophoresis. cDNA synthesis was done using 2 μg of RNA and reverse transcription was performed using SUPERSCRIPT II Reverse Transcriptase (Invitrogen) and oligo(dT).

RT-PCRs were carried out in 25 μl containing 1 μl of first-strand cDNA, 1× Taq DNA polymerase buffer (Invitrogen), 1 mM MgCl₂, 0.1 mM dNTPs, 1 unit of Taq DNA polymerase (Invitrogen) and 0.3 μM of each of the specific primers (Table 1). Primers were designed on different exons located preferentially at the 3′ end of the transcript using the Primer3 program developed by the Whitehead Institute for Biomedical Research. Amplification conditions were: initial denaturation for 4 min at 94° C. followed by 40 cycles of 45 sec at 94° C., 45 sec at specific annealing temperature determined by Oligotech® and 1 min at 72° C., with a final extension step of 6 min at 72° C. PCR products were analyzed on 8% silver-stained polyacrylamide gels and were also sequenced to confirm their specificity by using the Dynamic™ ET Terminator Cycle Sequencing Kit (Amersham Biosciences) available for sequencing on ABI Prism 3100 DNA Sequencer (Applied Biosystems).

TABLE 1
Primers used for expression analysis of CT1.1
(CTSP-5), CT1.11 (CTSP-6), CT1.19 (CTSP-7),
CT1.26 (CTSP-8), and CT1.29 (CTSP-9).

| CT/ | | SEQ ID | PCR |
|---|---|---|---|
| CTSP | Primer sequences | No | fragment |
| CT1.1/ | F 5′TGCTGCATGTGTTCCTCAG3′ | 1 | 112 bp |
| CTSP-5 | R 5′AGCTTCATCACTGACTGCC3′ | 2 | |
| CT1.11/ | F 5′GGAGTGAAGAGGAGTGGCTG3′ | 3 | 291 bp |
| CTSP-6 | R 5′TTGGCCACCTCTGCATAAC3′ | 4 | |
| CT1.19/ | F 5′CGAGAGCGAGGATGATGG3′ | 5 | 143 bp |
| CTSP-7 | R 5′GGAGCTCCTGGACCAATG3′ | 6 | |
| CT1.26/ | F 5′GATCAGGTGATGCAGAACTC3′ | 7 | 347 bp |
| CTSP-8 | R 5′GGTTTTACCTCCATTGTG3′ | 8 | |
| CT1.29/ | F 5′CCGAAAAGGGAGAAGAGGC3′ | 9 | 194 pb |
| CTSP-9 | R 5′GATCGTTGTCTGTATTGCTGC3′ | 10 | |

Extension of Partial cDNA Sequences Corresponding to CT1.11 (CTSP-6) and CT1.29 (CTSP-9) by RACE (Rapid Amplification of cDNA Ends).

5′-RACE and 3′-RACE were performed on normal testis poly(A)⁺ RNA by using the Marathon cDNA Amplification Kit (Clontech, Mountain View, Calif.). Amplifications reactions were performed in 25 μl by using 2.5 μl of cDNA, 0.2 mM dNTPs, 0.2 μM CTs specific primer (Table 2), 0.2 μM adaptor primer and 1 unit of Advantage Taq DNA polymerase. PCR conditions were: 5 cycles of 5 sec at 94° C. and 3 min at 72° C., 5 cycles of 5 sec at 94° C., 10 sec at 70° C. and 3 min at 72° C. and 25 cycles of 5 sec at 94° C., 10 sec at 68° C. and 3 min at 72° C. Nested PCRs were carried out by using 5 μl of the first reaction product diluted 50× and internal primers (Table 2). Amplification conditions consisted on 20 cycles of 5 sec at 94° C., 10 sec at 68° C. and 3 min at 72° C. PCR fragments were cloned by using the TA Cloning Kit (Invitrogen) and sequenced as described above.

Since the sequence corresponding to CT1.29 (CTSP-9) had already a polyA signal and a polyA tail, we have just performed 5′-RACE to extent the initial sequence of this candidate.

TABLE 2
Primers used for RACE experiments of CT1.11
(CTSP-6) and CT1.29 (CTSP-9). CT1.11 (CTSP-6)
primers named as F and FN were used on
5′-RACE experiments and primers identified as
R and RN were used on 3′-RACE.

| CT/CTSP | First Reaction | SEQ ID No |
|---|---|---|
| CT1.11/ | F-5′GATTTTCCAGACCTTGTCCAAGCTCC3′ | 11 |
| CTSP-6 | R-5′CACTCCTCTTCACTCCTTTCTATGGC3′ | 12 |
| CT1.29/ | R-5′CACACAGAATGCCGCTCGCTAGGAG3′ | 13 |
| CTSP-9 | | |
| CT/CTSP | Nested PCR | SEQ ID No |
| CT1.11/ | FN-5′GTGGCCAACTGAGCTGCAGACTTCCC3′ | 14 |
| CTSP-6 | RN-5′CTGCCTTGGATGGTGACATGAGTG3′ | 15 |
| CT1.29/ | RN-5′GATGTTTCCTCTTAGGCCTCTTCTCC3′ | 16 |
| CTSP-9 | | |

### Characterization of Novel Cancer Testis Antigens

### Transcriptome Database

The database (Map4) used in this work contains data obtained from alignments of all cDNA sequences to the human genome sequence. The Map4 database contains 3,475,517 expressed sequences of which 52,903 represent full-length cDNA sequences and 3,422,614 represent expressed sequence tags (EST). All these sequences were grouped generating 318,275 clusters, 21,306 containing at least one full-length cDNA sequence.

In order to define the criteria to be used in the cluster selection and also find possible limitations of our strategy, we first evaluated the EST content of clusters corresponding to 20 known CT antigens (MAGE-A1, MAGE-A3, MAGE-A10, MAGE-B1, CT7/MAGE-C1, CT10/MAGE-E1, NY-ESO-1, SSX-1, SSX-2, SSX-4, CT16/PAGE-5, HOM-TES-85, BRDT/CT9, CTp11/SPANX, OY-TES-1, CTAGE, CT15/Fertilin Beta, TRAG-3, IL-13RA2, HCA661/E2F-like). These clusters had at least one corresponding EST and 50% of them were composed by no more than ten sequences demonstrating the low level expression of these antigens. In addition, sequences corresponding to NY-ESO-1 and BRDT/CT9 aligned in more than one location on the genome suggesting an event of gene duplication that results in two or more identical copies with identical coding sequences. Therefore the 20 antigens were represented in Map4 database by 23 clusters.

It is noteworthy that only 10% of these clusters were composed of ESTs derived from testis and tumoral cDNA libraries demonstrating the characteristic expression pattern of CT antigens. The majority of these clusters contained ESTs from unknown cDNA libraries and 85% also contained ESTs derived from normal tissues. Besides this, only 39.1% contained ESTs derived from testis cDNA libraries showing the reduced number of sequences derived from this tissue and that was submitted to public databases. Of the set of 3,422,614 expressed sequence tags presented in our database, only 35,949 (1.05%) were derived from testis. Based on these observations, we concluded that using the presence of ESTs derived from testis cDNA libraries to select candidate clusters would be considered a restricted criterion and we decided to use it as a nonobligatory condition.

Furthermore, the great number of ESTs derived from normal cDNA libraries presented in the clusters corresponding to known CT antigens highlighted two problems in our database. First, the presence of annotation errors due to the absence of a common structured vocabulary to describe sequences submitted to GenBank which could falsely classify a library as derived from normal tissue. Second, multiple alignments of expressed sequences in the human genome as a consequence of the presence of gene families with a similarity higher than 95% among its members. Based on the alignment criterion of Map4, ESTs corresponding to a specific member of a gene family could also align in the clusters corresponding to the other members. Since some CT gene families are composed by members with a ubiquitous expression that do not correspond to a CT antigen, ESTs derived from normal cDNA libraries corresponding to these members would also be presented in clusters of CT antigens. In order to eliminate the presence of multiple alignments of expressed sequences in the human genome, we created the Map4_best hits database (Map4_bh). Sequences mapping to more than one location on the genome were given a score for alignment quality that was associated with a higher identity over a longer alignment. Only the sequences with the highest scores were kept reducing the total number of clusters from 318,275 in Map4 to 276,586 in Map4_bh. The EST content of clusters corresponding to the 20 known CT antigens were evaluated in Map4_bh demonstrating that sequences that aligned to different members of a gene family in Map4 now aligned in only one region of the genome sequence. In this way the number of expressed sequences derived from normal cDNA libraries was reduced in the clusters of CT antigens.

### Cluster Selection and Manual Inspection

Considering the tissue origin of the expressed sequences corresponding to each gene it was possible to define an in silico expression pattern and to select novel CT antigen candidates. Clusters composed of spliced ESTs derived from testis and/or tumoral cDNA libraries were automatically selected from Map4_bh and divided in three groups: group 1 corresponding to clusters composed of ESTs derived from only testis cDNA libraries; group 2 corresponding to clusters composed of ESTs derived from testis and tumoral cDNA libraries; group 3 corresponding to clusters composed of ESTs derived only from tumoral cDNA libraries. By using these criteria, a total of 1,184 candidate clusters were initially selected and manually inspected to confirm the presence of conserved acceptor and donor splicing sites (GT/AG) and to exclude clusters that correspond to know CT antigens. Following this procedure, a subset of 70 candidate clusters was initially selected for experimental validation. Most of the clusters automatically selected correspond to group 1 and consequently the majority of clusters selected for experimental validation are from the same group.

### Expression Analysis

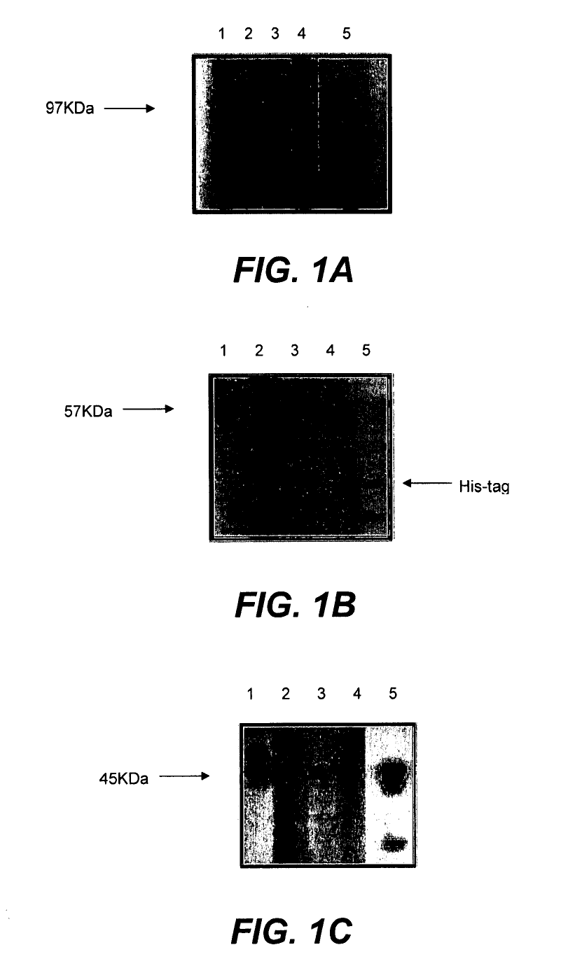

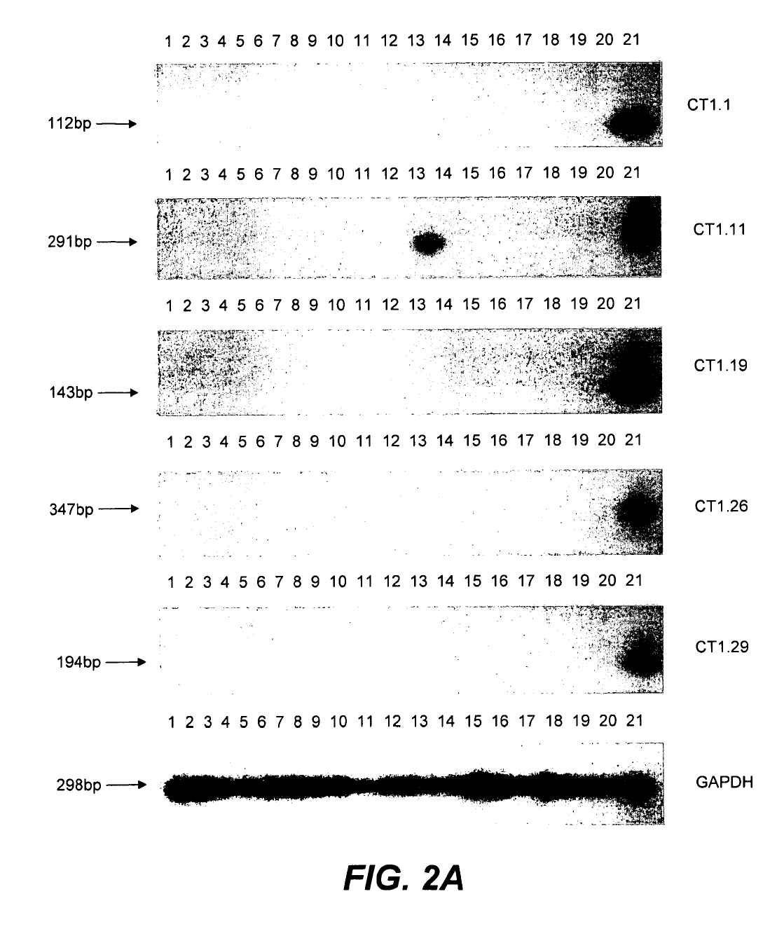

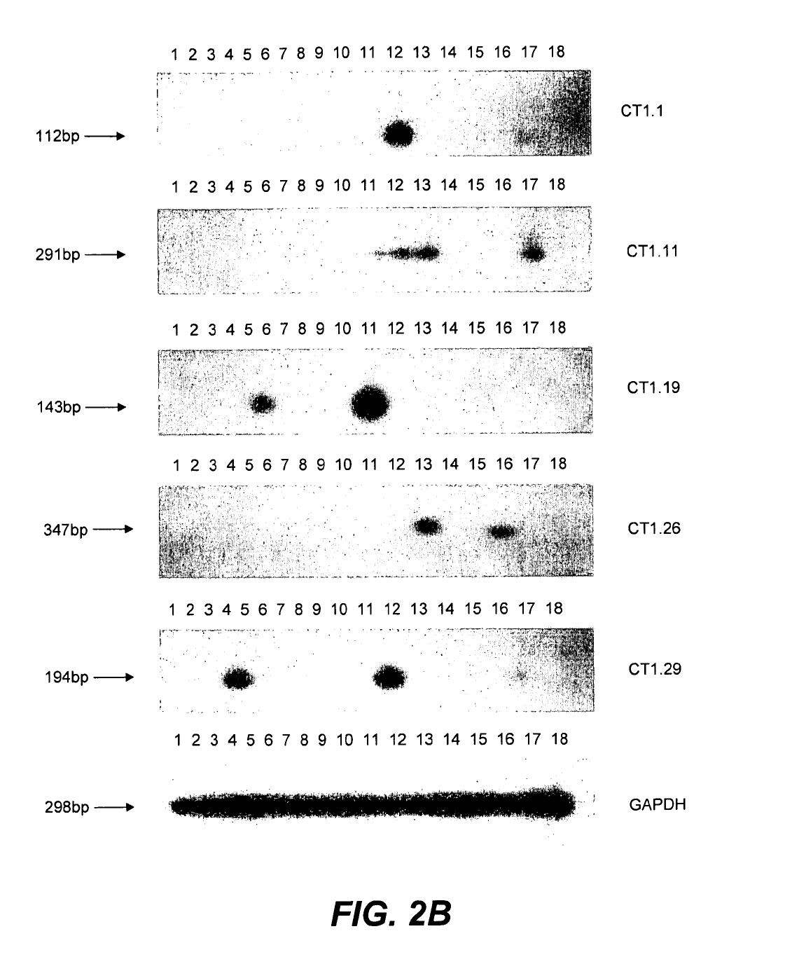

Following cluster selection and manual inspection, primers for RT-PCR validation of each candidate were manually designed using the Primer3 program. The experimental validation of the expression pattern was carried out in 21 normal tissues, 17 tumor cell lines and 160 samples derived from 9 different types of tumors. First, mRNA expression of each cluster was examined in normal testis. Few modifications of the standard RT-PCR protocol were applied when a positive amplification was not achieved including annealing temperature, MgCl₂ concentration and addition of PCR enhancers such as 1M betaine. Of the set of 70 selected clusters, seven candidates were expressed exclusively in testis and the expression of five of them (CT1.1 (CTSP-5), CT1.11 (CTSP-6), CT1.19 (CTSP-7), CT1.26 (CTSP-8) and CT1.29 (CTSP-9)) was also detected in several tumor cell lines (FIG. 2). Candidate CT1.11 (CTSP-6) also showed an expression in placenta but since it is a common feature of CT antigens expression the candidate was not excluded. Furthermore we were able to identify five candidates that were expressed in testis and tissues from nervous system (brain, fetal brain, cerebellum and spinal cord) characterizing them as novel candidates to cancer/testis/brain antigens (CTB). These antigens are a category of CT antigens and are expressed within normal tissues only in testis and brain and in different histological types of tumors. The expression of two of them was also detected in cell lines validating them as novel CTB antigens.

RT-PCR products of the five final CT candidates were transferred to nylon membranes and hybridized with a cDNA probe corresponding to each PCR product (FIG. 2). Southern blot analysis was applied to confirm the expression data since the sensitivity and specificity of this assay is higher than RT-PCR.

The expression analysis of the 5 CT candidates ((CT1.1 (CTSP-5), CT1.11 (CTSP-6), CT1.19 (CTSP-7), CT1.26 (CTSP-8) and CT1.29 (CTSP-9)) was then carried out in 160 samples derived from 9 different histological types of tumors (see Table 5 and Table 6).

### CT1.1, CTSP-5 (NM_{—}173493)=PASD1.

Among normal tissues, CT1.1 (CTSP-5) expression is restricted to testis. Its expression is also detected in 41% ( 65/160) of tumor samples and the highest frequency of expression was observed in glioblastomas (70%) (see Table 5). This candidate corresponds to PASD1 gene [PAS domain containing protein 1 (NM_{—}173493)] (see Table 6) maps to the telomeric end of the q arm of chromosome X between q24-28 and it has 4,120 bp organized in 15 exons. This gene was identified as PASD1_v1 as it has an alternatively spliced variant (PASD1_v2) that corresponds to 2,850 bp. A comparison of their sequences with the human genome sequence demonstrated that the 1.27 Kb sequence corresponds to intron 14 that is retained within the PASD1_v1 transcript. Both isoforms encode predicted proteins with the same N-terminal sequence and while the retained intron in PASD1_v1 introduces a stop codon after amino acid 639, PASD1_v2 encodes an additional 134 amino acids at the C-terminus. PASD1 gene expression data by RT-PCR confirmed the expression of PASD1_v1 in DLBCL derived cell lines while PASD1_v2 appeared to be expressed only in the cell lines derived from a poor prognosis subtype of DLBCL. Therefore the expression of the longer PASD1_v2 protein may be considered a marker for the identification of high risk DLBCL patients. This gene was characterized as a novel CT antigen by Liggins et al. (Br J Cancer. 2004 Jul. 5; 91(1):141-9).

PASD1 predicted proteins have in common a PAS (Per/ARNT/Sim) domain, a putative leucine zipper and a nuclear localization signal that together suggest it encodes a transcription factor. Immunolabeling studies using antibodies anti-PASD1 showed a normal tissue expression restricted to nuclei of spermatogonia near the basal membrane in testicular tubules confirming the prediction of a nuclear localization signal.

### CT1.11, CTSP-6

CT1.11 (CTSP-6) is expressed in testis and placenta among normal tissues and in 65% ( 104/160) of tumor samples. The highest expression frequency was observed among lung tumors (93%) (see Table 5). It corresponds to the expressed sequence tag AI652043 and maps to chromosome 11p15.4. The corresponding full-length cDNA sequence and protein are not yet available. To extend the EST sequence we performed RACE experiments and through this approach we were able to generate four specific fragments corresponding to splicing variants. The extension sequences were deposited in the Genbank Database (accession nos. EF537578-EF537581) and the corresponding consensus sequences are CT1.11a (CTSP 6.1) (SEQ ID NO 24), CT1.11b (CTSP 6.2) (SEQ ID NO 25), CT1.11c (CTSP 6.3) (SEQ ID NO 26), CT1.11d (CTSP 6.4) (SEQ ID NO 27).

The nucleotide sequence of each CT was translated to amino acid sequence and the most probable ORF was considered the longest one. For CT1.11 (CTSP-6) we analyzed the sequences corresponding to each isoform and the longest ORF predicted for CT1.11a (CTSP-6.1) generated a 115 amino acid putative protein (SEQ ID NO 29). For the other three isoforms (CT1.11b (CTSP-6.2) (SEQ ID NO 30), CT1.11c (CTSP-6.3) (SEQ ID NO 31) and CT1.11d (CTSP-6.4) (SEQ ID NO 32)) the same ORF was predicted generating a 107 amino acids putative protein. Motif analysis of these ORFs identified only promiscuous phosphorylation sites.

### CT1.19, CTSP-7 (AF461259)=ASZ1=GASZ

CT1.19 (CTSP-7) is expressed only in testis among normal tissues and expression of this candidate can be detected in 20% ( 31/155) of tumor samples with the highest frequency among uterus tumors (50%) (see Table 5). The transcript corresponds to ASZ1 or GASZ gene (Germ cell-specific ankyrin, SAM and basic leucine zipper domain containing protein 1) (see Table 6) that maps to 7q31.2 and which transcript has 1,831 bp divided in 13 exons. It contains a 1,427 bp open reading frame which encodes a predicted protein of 475 amino acids composed of four ankyrin repeats, a SAM (Sterile Alpha Motif) and a bZIP domain. Immunohistochemistry showed that GASZ protein is localized to the cytoplasm of spermatocytes and oocytes at different stages. Based on its functional domains, GASZ may act as a signaling protein and/or transcriptional regulator during germ cell maturation and early embryogenesis.

### CT1.26, CTSP-8 (BC028710)=FAM46D

CT1.26 (CTSP-8) expression is restricted to testis among normal tissues. It is also expressed in 24% ( 38/160) of tumor samples and predominantly expressed in lung tumors (50%) (see Table 5). The transcript corresponds to FAM46D gene [Family with sequence similarity 46, member D (BC028710)] (see Table 6) and maps to the q21.1 region of chromosome X. The transcript contains 3,008 bp distributed in 3 exons and it has an open reading frame of 1,169 bp which encodes a predicted protein of 389 amino acids with no specific protein domain. The encoded protein does not have any known functional motif.

### CT1.29, CTSP-9 (AA451827)

Among normal tissues candidate CT1.29 (CTSP-9) expression is restricted to testis. Moreover, it is expressed in 18% ( 28/156) of the tumor samples being more frequently expressed in gastric tumors (33.3%) (see Table 5). It corresponds to the expressed sequence tag AA451827 and maps to chromosome Xq23 (see Table 6). The corresponding full-length cDNA sequence and protein are not yet available. An extension sequence was generated by RACE experiments and deposited in the Genbank Database (accession no. EF537582). The corresponding consensus sequence is CT1.29 (CTSP-9) (SEQ ID NO 28). The longest ORF in CT1.29 (CTSP-9) corresponds to a 88 amino acid putative protein and the motif analysis of the sequence identified only phosphorylation sites.

TABLE 5
Frequency of mRNA expression of the five CT antigens candidates identified by the
in silico approach in tumor samples.

| | CT1.1 | | CT1.11 | | CT1.19 | | CT1.26 | | CT1.29 | |
|---|---|---|---|---|---|---|---|---|---|---|
| Tumor | (CTSP-5) | | (CTSP-6) | | (CTSP-7) | | (CTSP-8) | | (CTSP-9) | |
| Colon | 7/18 | (39%) | 15/18 | (83.3%) | 4/18 | (22.2%) | 4/18 | (22.2%) | 4/18 | (22.2%) |
| Stomach | 4/9 | (44.5%) | 5/9 | (55.5%) | 0/9 | (0%) | 1/9 | (11.2%) | 3/9 | (33.3%) |
| Glioblastoma | 9/13 | (70%) | 10/13 | (77%) | 1/13 | (7.7%) | 1/13 | (7.7%) | 0/13 | (0%) |
| Breast | 10/24 | (41.7%) | 14/24 | (58.3%) | 5/22 | (22.7%) | 4/24 | (16.67%) | 5/21 | (23.8%) |
| Melanoma | 8/18 | (44.5%) | 10/18 | (55.5%) | 3/18 | (16.7%) | 5/18 | (27.8%) | 4/18 | (22.2%) |
| Prostate | 6/21 | (28.5%) | 7/21 | (33.3%) | 2/21 | (9.5%) | 7/21 | (33.3%) | 3/21 | (12.3%) |
| Lung | 4/14 | (28.5%) | 13/14 | (93%) | 4/14 | (28.5%) | 7/14 | (50%) | 3/14 | (21.4%) |
| Uterus | 11/19 | (58%) | 15/19 | (79%) | 9/18 | (50%) | 7/19 | (37%) | 5/18 | (27.77%) |
| Thyroid | 6/24 | (25%) | 15/24 | (62.5%) | 3/22 | (13.7%) | 2/24 | (8.3%) | 1/24 | (4.16%) |
| Total | 65/160 | (41%) | 104/160 | (65%) | 31/155 | (20%) | 38/160 | (24%) | 28/156 | (18%) |

For the expression analysis of CT1.1 (CTSP-5) and CT1.11 (CTSP-6), primers were constructed in common exons among the isoforms and the results represent the accumulated expression of all isoforms.

TABLE 6
Gene name and chromosome location corresponding to
each of the five CT candidates.

| Candidate | Reference Sequence | Gene name | Chromosome |
|---|---|---|---|
| CT1.1 = CTSP-5 | BI458651 | PASD1 | Xq28 |
| CT1.11 = CTSP-6 | AI652043 | — | 11p15.4 |
| CT1.19 = CTSP-7 | BG771896 | ASZ1 | 7q31.2 |
| CT1.26 = CTSP-8 | BG722950 | FAM46D | Xq21.1 |
| CT1.29 = CTSP-9 | AA451827 | CXorf61 | Xq23 |

### Example 2

### Analysis of the Presence of a Humoral Immune Response Against CT1.1 (CTSP-5), CT1.19 (CTSP-7), CT1.26 (CTSP-8) in Cancer Patients

The candidates CT1.1 (CTSP-5), CT1.19 (CTSP-7), CT1.26 (CTSP-8) that have a full-length sequence available were chosen to evaluate the presence of a humoral response in cancer patients. The longest ORFs of CT1.1 (CTSP-5) (773 amino acids), CT1.19 (CTSP-7) (475 amino acids) and CT1.26 (CTSP-8) (389 amino acids) were amplified from normal testis cDNA by using specific primers (Table 3).

### CT1.1 (CTSP-5) Recombinant Protein

CT1.1 (CTSP-5) longest ORF (773aa) was amplified from normal testis cDNA using specific primers PTN101F and PTN101R (Table 3). A reverse primer PROT101R was constructed after PTN101R to be used in a first reaction in order to facilitate the fragment amplification of 2,322 bp. The PCR product was digested with EcoRI and HindIII and cloned into the expression vector pET28a (Stratagene, La Jolla, Calif.) (Table 4). After sequencing, the recombinant plasmid pET28a/CT1.1 was transformed into Escherichia coli BL-21 Rosetta and the CT1.1 recombinant protein was expressed and purified.

### CT1.19 (CTSP-7) Recombinant Protein

CT1.19 (CTSP-7) longest ORF (475aa) was amplified from normal testis cDNA using specific primers PTN802F32 and PTN802R32 (Table 3). The PCR product was digested with EcoRV and EcoRI and cloned into the expression vector pET32a (Stratagene, La Jolla, Calif.) (Table 4). After sequencing, the recombinant plasmid pET32a/CT1.19 was transformed into Escherichia coli BL-21 Rosetta and the CT1.19 recombinant protein was expressed and purified.

### CT1.26 (CTSP-8) Recombinant Protein

CT1.26 (CTSP-8) longest ORF (389aa) was amplified from normal testis cDNA using specific primers PTN809F and PTN809R (Table 3). The PCR product was digested with EcoRI and HindIII and cloned into the expression vector pET28a (Stratagene, La Jolla, Calif.) (Table 4). After sequencing, the recombinant plasmid pET28a/CT1.26 was transformed into Escherichia coli BL-21 Rosetta and the CT1.26 recombinant protein was expressed and purified.

TABLE 3
Primers used for the amplification of CT1.1 (CTSP-5), CT1.19
(CTSP-7), and CT1.26 (CTSP-8) ORF sequences. Primers PTN101F and
PROT101R were used in the first reaction of CT1.1 (CTSP-5) ORF
amplification while PTN101F and PTN101R were used in the
second reaction.

| CT/ | | SEQ ID | PCR |
|---|---|---|---|
| CTSP | Primers sequences | No | fragment |
| CT1.1/ | PTN101F 5′ CGGAATTCATGAAGATGAGAGGGGAAAAG 3′ | 17 | 1st reaction |
| CTSP-5 | PTN101R 5′ CCCAAGCTTTTAGCACGGCTTATTTGAGTC 3′ | 18 | −2.387 bp |
| | PROT101R 5′ CATGCAGACCTCATTGGCCTG 3′ | 19 | 2nd reaction |
| | | | −2.322 bp |
| CT1.19/ | PTN802F32 5′ GATATCATGGCGGCGAGCGCGCTGCGA 3′ | 20 | 1.428 bp |
| CTSP-7 | PTN802R32 5′ CGGAATTCTTATTTCCTCTGGAAAGTTAG 3′ | 21 | |
| CT1.26/ | PTN809F 5′ CGGAATTCATGTCTGAAATCAGATTCACC 3′ | 22 | 1.170 bp |
| CTSP-8 | PTN809R 5′ CCCAAGCTTTTAACTCATACCATTTGATCC 3′ | 23 | |

TABLE 4
Restriction enzymes used for digestion of respective PCR products
and expression vectors used to clone each of the amplicons.

| | Restriction | |
|---|---|---|
| CT/CTSP | Enzymes | Expression Vector |
| CT1.1/CTSP-5 | EcoRI/HindIII | pET28a (Stratagene, La Jolla, CA) |
| CT1.19/CTSP-7 | EcoRV/EcoRI | pET32a (Stratagene, La Jolla, CA) |
| CT1.26/CTSP-8 | EcoRI/HindIII | pET28a (Stratagene, La Jolla, CA) |

### Purification and Protein Analysis

After induction of Escherichia coli BL-21 Rosetta with 0.4 mM isopropyl β-D-thiogalactoside at 37° C. for 4 h, the CT1.1, CT1.19, CT1.26 recombinant proteins fused with a 6His-tag were purified by Ni²⁺ affinity chromatography using a NiNTA agarose resin (Invitrogen). The purified proteins were analyzed by Western blot using an anti-His-tag monoclonal antibody (Amersham Biosciences, Piscataway, N.J.) to confirm the purification specificity. Briefly, five hundred nanograms of CT1.1, CT1.19, CT1.26 recombinant protein were fractioned on 12% SDS/PAGE gel electrophoresis and transferred to Hybond-P PVDF membranes. After blocking with PBS solution containing 5% low-fat milk, the membranes were incubated for 60 minutes at room temperature with anti-His-tag antibody at a 1:10,000 dilution. CT1.1, CT1.19, CT1.26 protein was detected by incubation with rabbit anti-mouse IgG HRP-conjugate (Amersham Biosciences) and visualized with ECL™ Western Blotting Detection Reagents (Amersham Biosciences) (FIG. 1a-c; lane 5).

### Antibody Response in Cancer Patients

Plasmas were obtained from patients treated at Hospital A. C. Camargo. All samples were collected after explicit informed consent and with local ethical committee approval. In addition, plasma samples from 30 healthy individuals were collected from blood donors at the Hospital A. C. Camargo Blood Center.

Antibodies in the plasma against CT1.1 (CTSP-5), CT1.19 (CTSP-7) and CT1.26 (CTSP-8) recombinant proteins were detected by Western blot (FIG. 1a-c; lanes 1-4). Five hundred nanograms of purified recombinant proteins were fractioned on 12% SDS/PAGE and transferred to Hybond-P PVDF membranes. After blocking with PBS solution containing 5% low-fat milk, membranes were incubated for 60 minutes at room temperature with plasma from cancer patients (FIG. 1a-c; lanes 1 and 2) or healthy individuals (FIG. 1a-c; lanes 3 and 4) at a 1:100 dilution in 1% low-fat milk. Plasma antibodies binding to CT1.1 (CTSP-5) (FIG. 1a), CT1.19 (CTSP-7) (FIG. 1b) and CT1.26 (CTSP-8) (FIG. 1c) proteins were detected by incubation with goat anti-human IgG HRP conjugate (Amersham Biosciences) and visualized with ECL Western Blotting Detection Reagents.

Humoral immunity is the production of antibody in response to antigen. The B-lymphocyte is the primary cell responsible for producing antibody. However, this cell is regulated by T-lymphocytes and macrophages. Many methods are available to assess humoral immune responses. A multitude of immunoassays have been developed for enumeration of serum antibody. Some of these are immunodiffusion, complement fixation, serum neutralization, hemagglutination, radioimmunoassay and enzyme-linked immunosorbent assay (ELISA). This technique is highly sensitive and can be automated and hence can easily be incorporated into the drug efficacy testing programs of industry.

Detection of antibody-forming cells distinguishes between effect on antibody production compared to degradation of preformed antibody. Other available methods are measurement of surface receptor (Fc and complement) activity on B-cells. Mitogens (T-independent) have also been regarded as a measurement for humoral immunity.

To evaluate the presence of antibodies against the proteins corresponding to the CT candidates in plasma from cancer patients, we established an ELISA assay by using recombinant His-tagged proteins. His-tagged unrelated recombinant proteins from sugarcane, expressed and purified under the same conditions as the recombinant CT proteins, were used to correct for background binding. Three (CT1.1, CT1.19 and CT1.26) of the five CT candidates have a full-length sequence available in public databases and were chosen to evaluate the presence of humoral response in cancer patients (Table 7).

Antibodies against CT1.1, CT1.19 and CT1.26 recombinant proteins were determined by direct ELISA. Microplates were coated with 50 μl/well of each recombinant protein diluted (5.0 μg/ml) in PBS buffer pH 7.4 overnight at 4° C. Wells were blocked with 5% (w/v) milk powder in PBS buffer pH 7.4, washed 3 times with PBS-Tween (0.05%), and incubated with 50 μl serum diluted 1:50 in dilution buffer (1% (w/v) milk powder in PBS) for 2 hr at 37° C. After washing, 50/21 goat anti-human IgG conjugated to horseradish peroxidase (Sigma) diluted 1:20,000 in PBS was used as secondary antibody for 1 hr at 37° C., followed by washing. Bound secondary antibody was visualized by the HRP enzymatic reaction using OPD tablets (DAKO). Absorbance at 492 m was read on a SpectroCount analyzer and expressed as optical density (OD) values. All serum samples were analyzed in triplicates and corrected for background binding to an irrelevant protein from sugar cane expressed in the same expression system as the recombinant proteins. ELISA cut off was established as the mean+2SD of healthy blood donors.

Antibodies against recombinant His-tagged CT1.1 (97 KDa), CT1.19 (57 KDa) and CT1.26 (44 KDa) were detected in 19 plasma samples from lung cancer patients, 31 plasma samples from uterus cancer patients, 24 plasma samples from colon cancer patients, 25 plasma samples from glioblastoma patients and 30 plasma from healthy blood donors (see Table 7). Anti-CT1.1 antibodies [transcript corresponding to PASD1 gene (PAS domain containing protein 1)] were observed in 29% ( 9/31) of plasma samples from patients with uterus tumor; 5.2% ( 1/19) of plasma from patients with lung cancer; 12.5% ( 3/24) of plasma samples from patients with colon cancer and 30.4% ( 7/25) of plasma samples from patients with glioblastoma. The frequency of anti-CT1.19 antibodies [transcript corresponding to ASZ1 or GASZ gene (Germ cell-specific ankyrin, SAM and basic leucine zipper domain containing protein 1)] observed in plasma samples from patients with uterus tumor was 13% ( 4/31) while among patients with lung cancer the frequency was 31.6% ( 6/19). We also observed anti-CT1.19 antibodies in 20.8% ( 5/24) of plasma samples from patients with colon cancer and 4% ( 1/25) of plasma samples from patients with glioblastoma. Finally, the frequency of anti-CT1.26 antibodies, that corresponds to FAM46D gene (Family with sequence similarity 46, member D) was 13% ( 4/31) among patients with uterus tumors; 5.2% ( 1/19) among patients with lung cancer and 4.1% ( 1/24) among patients with colon cancer. We did not find antibodies against CT1.26 in plasmas from glioblastoma patients.

TABLE 7
Frequency of anti-CT1.1, -CT1.19 and -CT1.26 antibodies in
plasma samples from cancer patients.

| | Antibody response (%) | | |
|---|---|---|---|
| | CT1.1 | CT1.19 | CT1.26 |
| Tumor | (CTSP-5) | (CTSP-7) | (CTSP-8) |
| Uterus | 9/31 (29.0) | 4/31 (13.0) | 4/31 (13.0) |
| Lung | 1/19 (5.2) | 6/19 (31.6) | 1/19 (5.2) |
| Colon | 3/24 (12.5) | 5/24 (20.8) | 1/24 (4.1) |
| Glioblastoma | 7/25 (30.4) | 1/25 (4.0) | 0/25 (0.0) |
| Total | 20/99 (20.2) | 16/99 (16.1) | 6/99 (6.0) |

Using SEREX, Guinn et al. (2005) found that 35% ( 6/17) of sera from diagnosed acute myeloid leukaemia (AML) patients, 6% ( 1/16) of sera from chronic myeloid leukaemia (CML) patients, 10% ( 1/10) of sera from DLBCL patients and none of the 18 normal donor sera were reactive with PASD1_v2 antigen, the same isoform we used to produce the CT1.1 recombinant protein. Moreover, Guinn et al. (2005) demonstrated that monocyte-derived dendritic cells electroporated with PASD1_V2 mRNA could stimulate autologous T-cells to proliferate. This study together with our data from RT-PCR expression and ELISA assays suggest that PASD1 may be a promising target for cancer immunotherapy.

It is noteworthy that the frequencies of anti-CT1.1 [20.2% ( 20/99)] and anti-CT1.19 [16.1% ( 16/99)] antibodies observed in plasma from cancer patients were impressive when compared to immunogenicity data from other CTs. At present CT1.1 is the most immunogenic among CT antigens eliciting a humoral immune response in 20% of prostate cancer patients. Humoral immune response to CT1.26 [6% ( 6/99)] in cancer patients is in common to other CT antigens occurring in no more than 10% of the cancer patients.

Several clinical trials involving peptides from different CT antigens such as MAGE-A3 and NY-ESO-1 are ongoing. Over 34 trials with different NY-ESO-1 vaccine preparations, such as NY-ESO-1 peptides or even the protein, have been conducted and all can induce strong humoral and cellular immunity in patients. The NY-ESO-1 Protein/ISCOMATRIX vaccine showed evidence for possible therapeutic benefits since circulating specific CD4⁺ and CD8⁺ T cells were detected in patients with advanced NY-ESO-1 expressing tumors and a Phase II randomized trial in now ongoing.

### Example 3

### CT1.1 as a Diagnostic Tool for Glioblastoma

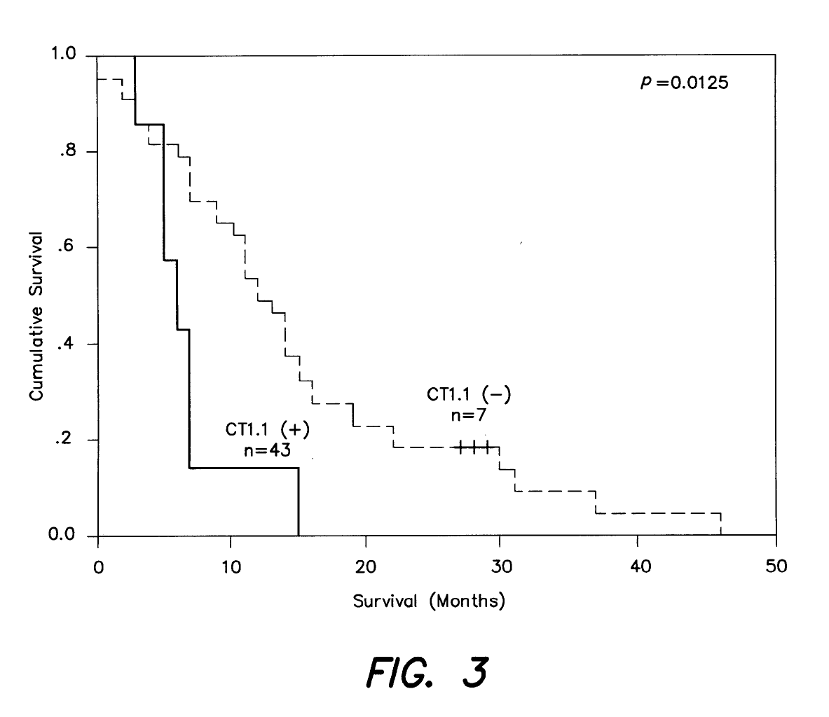

The presence of antibodies against CT1.1 (CTSP-5) in serum (humoral response) from a collection of 50 glioblastoma patients was tested by ELISA. We observed the presence of anti-CT1.1 antibodies in 16% of the plasma samples and a survival curve indicated that the presence of anti-CT1.1 antibodies was significantly associated with a poor prognosis (p=0.0125) (FIG. 3). Thus, the presence of CT antibodies, such as e.g. CT1.1, in serum can be related to outcome of disease progression, thus providing direction for therapeutic targeting. The presence of CT antibodies in tumors may be used to identify patients that may be responsive to treatment with monoclonal antibody specific for CTs (monoclonal antibody therapy). Humoral immunity to a specific cancer antigen may provide prognostic information but can also identify individuals capable of mounting an immune response to that antigen and identifying patients who might benefit from vaccine treatments that target the specific antigen (see Carter D, Clinical Cancer Research Vol. 9, 749-754, February 2003).

Expression analysis of CT1.1 was carried out (as described herein) in 40 samples derived from different brain tumor stages by RT-PCR. Astrocytomas are gliomas of astrocytic origin and constitute the most common type of primary brain tumor. They are classified, according to the World Health Organization (WHO), into pilocytic astrocytomas (PA) (grade I, GI), low grade astrocytomas (grade II), anaplastic astrocytomas (grade III) and glioblastoma multiforme (GBM) (grade IV). GBM is resistant to conventional therapies and survival time is usually shorter than 12 months. CT1.1 is not expressed in normal brain (see FIG. 2). Based on this analysis, we found that a higher expression of CT1.1 was associated with a higher tumor grade (Table 8).

TABLE 8
Frequency of mRNA expression of CT1.1 relative to normal tissue.

| Samples | Frequency of mRNA expression |
|---|---|
| Pilocytic Astrocytoma | 10% (1/10) |
| Astrocytoma grade II | 30% (3/10) |
| Astrocytoma grade III | 40% (4/10) |
| Glioblastoma | 50% (5/10) |

The data suggest that CT1.1 is a good marker for tumor progression and a marker for poor prognosis in glioblastoma, and may also constitute an important therapeutic target.

### Sequences

The following is a listing of the sequences identified:

CT1.11a (CTSP-6.1)
(SEQ ID NO. 24)
CTTTGGTCTAGAGCAAGTGTTGGCGAACTATTCTGTAAAGGGCTAGATAGTAAATGTCTGAGGTTCGTAGGATCA
TATGGTGTCTGTCACAACTACTCGACTCTGCCATTGCAGTGGAAAAGCAGCCAAGGACAATGGGTCTTCAGACCT
GTAGATGGGCTCTTCTTGGACATTCTGGAGTCCCTGGGGGAAAGGGAAGTCTGAAGCTCAGTTGGCCACCTCTGC
ATAACAAATACCGAGAGCACTGCAAGTGGAGCTTGGACAAGGTCTGGAAAATCAGCCTTCTTCTGGATGCCTCCA
GCCTTTGCTATCATCTGCCTAGGAGGTTGGAAGTTACTCAAGCGGCTCAAGGAAAAGTCCCTATTTCTGTTGCAG
TTGATTTGGAGTTATGTGATGTGTAGCCCATCAGAAGTAAGCAGAAAACTTTGTGCCGTTTTCTATCCTGGAAAT
AACACCCCCGCCCCCTGGCCCCGCTGCATTATCAGCCACTCCTCTTCACTCCTTTCTATGGCTCCAGAAGTGAAG
AGCTCTGAAATGCCTAAATTGCCAGATGGGAACATCTTTACTGCTTGGAGAAAGCCACACAGGAGTTACCTGACC
TGCCTTGGATGGTGACATGAGTGAGAAATAAACCTTTGTTGGGTTAGACTGCTAAA
CT1.11b (CTSP-6.2)
(SEQ ID NO. 25)
AGAGCCTCTCTGCATGAGATAAGGAAAGGAATTTATGTCTGACTTTGGAGAGATTGGATGGAGAAGAAAGAACAG
AGGAAAGAGAAGACAGAAGGAAGACAGAAGAACCAGAGGAGCATAACCTTCAGACCTGTAGATGGGCTCTTCTTG
GACATTCTGGAGTCCCTGGGGGAAAGGGAAGTCTGAAGCTCAGTTGGCCACCTCTGCATAACAAATACCGAGAGC
ACTGCAAGTGGAGCTTGGACAAGGTCTGGAAAATCAGCCTTCTTCTGGATGCCTCCAGCCTTTGCTATCATCTGC
CTAGGAGGTTGGAAGTTACTCAAGCGGCTCAAGGAAAAGTCCCTATTTCTGTTGCAGTTGATTTGGAGTTATGTG
ATGTGTAGCCCATCAGAAGTAAGCAGAAAACTTTGTGCCGTTTTCTATCCTGGAAATAACACCCCCGCCCCCTGG
CCCCGCTGCATTATCAGCCACTCCTCTTCACTCCTTTCTATGGCTCCAGAAGTGAAGAGCTCTGAAATGCCTAAA
TTGCCAGATGGGAACATCTTTACTGCTTGGAGAAAGCCACACAGGAGTTACCTGACCTGCCTTGGATGGTGACAT
GAGTGAGAAATAAACCTTTGTTGGGTTAGACTGCTAAA
CT1.11c (CTSP-6.3)
(SEQ ID NO. 26)
ACCTCAGCCTCCCAAGTAGCTGGGACTCCAAGTGGGTGCCACTACAGCCATGGTGTCTTTAACTGAGATATGAAG
ATCACAAGCAGAGAAGCTTCCGAGTTCAGGGAGGAAATCCAGAGTTCGGATTTGAGCATGTTAAGTTTGAGATCT
CCGTTAGACATCCACGAAGGTCAGGTAGGCACTTGAATAAATGAGACTGGAACTCAGAAGAGGCTTGGGCTGGAG
CGAAAGCTGAATTGTCAGCATATGGAGAATAGAACCTTCAGACCTGTAGATGGGCTCTTCTTGGACATTCTGGAG
TCCCTGGGGGAAAGGGAAGTCTGAAGCTCAGTTGGCCACCTCTGCATAACAAATACCGAGAGCACTGCAAGTGGA
GCTTGGACAAGGTCTGGAAAATCAGCCTTCTTCTGGATGCCTCCAGCCTTTGCTATCATCTGCCTAGGAGGTTGG
AAGTTACTCAAGCGGCTCAAGGAAAAGTCCCTATTTCTGTTGCAGTTGATTTGGAGTTATGTGATGTGTAGCCCA
TCAGAAGTAAGCAGAAAACTTTGTGCCGTTTTCTATCCTGGAAATAACACCCCCGCCCCCTGGCCCCGCTGCATT
ATCAGCCACTCCTCTTCACTCCTTTCTATGGCTCCAGAAGTGAAGAGCTCTGAAATGCCTAAATTGCCAGATGGG
AACATCTTTACTGCTTGGAGAAAGCCACACAGGAGTTACCTGACCTGCCTTGGATGGTGACATGAGTGAGAAATA
AACCTTTGTTGGGTTAGACTGCTAAA
CT1.11d (CTSP-6.4)
(SEQ ID NO. 27)
CTTTTTTCATTTTTTTCTTCACTTTTCTCCAGCCTTCCTCTCTCTCTCTCCTTCCTTCCTCCTTGCTTTCTTTAT
ATGCCTTATCTTCTGAGAAGGTTGACCCCAGTTCCGGCGATAGACGGACACTAAGCCCCCCTTGAGTGACTAGAA
AGGCAGGGGACATAGGAACAGTTAGGACATAAGGGAAGAGTAAAGAGAGGGACCCCTGATTTTGATTTCAGGGTG
CCCTGTTTGTCTCATCCAATATCCTGAAGACCCAAGATGGAGACCAGCTACTTGTCCCTTCCAAAAAGCTTCCCT
GGCCTTAATGTCAGTGCTTGTAGGTGATCTCAGAAACCTCCCTGGTGAAGAAAACCTGAAGACGGGGTCTTGCTC
TGTTGCCTGGCTGGAGTGCAGTGGTGCTAACATGACTCACTGCACCCTTGACCTCCTGGACTCAAGTGATCCTCC
CACCTCAGCCTCCCAAGTAGCTGGGACTCCAAGTGGGTGCCACTACAGCCAAACCTTCAGACCTGTAGATGGGCT
CTTCTTGGACATTCTGGAGTCCCTGGGGGAAAGGGAAGTCTGAAGCTCAGTTGGCCACCTCTGCATAACAAATAC
CGAGAGCACTGCAAGTGGAGCTTGGACAAGGTCTGGAAAATCAGCCTTCTTCTGGATGCCTCCAGCCTTTGCTAT
CATCTGCCTAGGAGGTTGGAAGTTACTCAAGCGGCTCAAGGAAAAGTCCCTATTTCTGTTGCAGTTGATTTGGAG
TTATGTGATGTGTAGCCCATCAGAAGTAAGCAGAAAACTTTGTGCCGTTTTCTATCCTGGAAATAACACCCCCGC
CCCCTGGCCCCGCTGCATTATCAGCCACTCCTCTTCACTCCTTTCTATGGCTCCAGAAGTGAAGAGCTCTGAAAT
GCCTAAATTGCCAGATGGGAACATCTTTACTGCTTGGAGAAAGCCACACAGGAGTTACCTGACCTGCCTTGGATG
GTGACATGAGTGAGAAATAAACCTTTGTTGGGTTAGACTGCTAA
CT1.29 (CTSP-9)
(SEQ ID NO. 28)
GCCCAACTGTGAGGGCGCGCGCTCTCAGCAGTATATAAAGCGGGACAACCCCAGAACATCCCAGTTGCACCAGGC
GATGCAAGACACATGGCAGTCTGGCCTGAAGTTTCCCAAAACAGGCTGACTAGGGGCCTACTGCTTCCCAACTAC
CAGCTGAGGGGGTCCGTCCCGAAAAGGGAGAAGAGGCCTAAGAGGAAACATCAACATCTTTTTACTCCTAGCGAG
CGGCATTCTGTGTGCCTTGATTGTCTTCTGGAAATATCGCTTTCAGGGAAACAATGGCGAAATGTCATCAGTTTC
AACTGCTTTTGCACTACTAAGACGCTTTTCTGGGTTAATTAGCAGCAATACAGACAACGATCTTTTATTCAACAA
CCTCTCTCGAGATATTTTAAATAATTTCTCACACTCGAAAAACATGCAGAAGCGACTATTGGCAAACCTGAAGAG
GGTGGAATACCAAATGGCTGAACTGGAATATTTTCTAGTTAGCGAGGGTTTGAGAGGTGCGTCAGGTCTCCAGAA
ATTCACCTCAAAAGCGTACAGGATGTAATGCCAGTGGTGGAAATCATTAAAGACACTTTGAGTAGAAAAAAAAAA
AAAAAAAAA

The sequences that are shown below indicate the ORF predicted for each transcript.

CT1.11a (CTSP-6.1)
(SEQ ID NO. 29)

| | |
|---|---|
| 54 | atgtctgaggttcgtaggatcatatggtgtctgtcacaactactc |
| | M S E V R R I I W C L S Q L L |
| 99 | gactctgccattgcagtggaaaagcagccaaggacaatgggtctt |
| | D S A I A V E K Q P R T M G L |
| 144 | cagacctgtagatgggctcttcttggacattctggagtccctggg |
| | Q T C R W A L L G H S G V P G |
| 189 | ggaaagggaagtctgaagctcagttggccacctctgcataacaaa |
| | G K G S L K L S W P P L H N K |
| 234 | taccgagagcactgcaagtggagcttggacaaggtctggaaaatc |
| | Y R E H C K W S L D K V W K I |
| 279 | agccttcttctggatgcctccagcctttgctatcatctgcctagg |
| | S L L L D A S S L C Y H L P R |
| 324 | aggttggaagttactcaagcggctcaaggaaaagtccctatttct |
| | R L E V T Q A A Q G K V P I S |
| 369 | gttgcagttgatttggagttatgtgatgtgtag 401 |
| | V A V D L E L C D V * |

CT1.11b (CTSP-6.2)
(SEQ ID NO. 30)

| | |
|---|---|
| 274 | atgcctccagcctttgctatcatctgcctaggaggttggaagtta |
| | M P P A F A I I C L G G W K L |
| 319 | ctcaagcggctcaaggaaaagtccctatttctgttgcagttgatt |
| | L K R L K E K S L F L L Q L I |
| 364 | tggagttatgtgatgtgtagcccatcagaagtaagcagaaaactt |
| | W S Y V M C S P S E V S R K L |
| 409 | tgtgccgttttctatcctggaaataacacccccgccccctggccc |
| | C A V F Y P G N N T P A P W P |
| 454 | cgctgcattatcagccactcctcttcactcctttctatggctcca |
| | R C I I S H S S S L L S M A P |
| 499 | gaagtgaagagctctgaaatgcctaaattgccagatgggaacatc |
| | E V K S S E M P K L P D G N I |
| 544 | tttactgcttggagaaagccacacaggagttacctgacctgcctt |
| | F T A W R K P H R S Y L T C L |
| 589 | ggatggtga 597 |
| | G W * |

CT1.11c (CTSP-6.3)
(SEQ ID NO. 31)

| | |
|---|---|
| 412 | atgcctccagcctttgctatcatctgcctaggaggttggaagtta |
| | M P P A F A I I C L G G W K L |
| 457 | ctcaagcggctcaaggaaaagtccctatttctgttgcagttgatt |
| | L K R L K E K S L F L L Q L I |
| 502 | tggagttatgtgatgtgtagcccatcagaagtaagcagaaaactt |
| | W S Y V M C S P S E V S R K L |
| 547 | tgtgccgttttctatcctggaaataacacccccgccccctggccc |
| | C A V F Y P G N N T P A P W P |
| 592 | cgctgcattatcagccactcctcttcactcctttctatggctcca |
| | R C I I S H S S S L L S M A P |
| 637 | gaagtgaagagctctgaaatgcctaaattgccagatgggaacatc |
| | E V K S S E M P K L P D G N I |
| 682 | tttactgcttggagaaagccacacaggagttacctgacctgcctt |
| | F T A W R K P H R S Y L T C L |
| 727 | ggatggtga 735 |
| | G W * |

CT1.11d (CTSP-6.4)
(SEQ ID NO. 32)

| | |
|---|---|
| 656 | atgcctccagcctttgctatcatctgcctaggaggttggaagtta |
| | M P P A F A I I C L G G W K L |
| 701 | ctcaagcggctcaaggaaaagtccctatttctgttgcagttgatt |
| | L K R L K E K S L F L L Q L I |
| 746 | tggagttatgtgatgtgtagcccatcagaagtaagcagaaaactt |
| | W S Y V M C S P S E V S R K L |
| 791 | tgtgccgttttctatcctggaaataacacccccgccccctggccc |
| | C A V F Y P G N N T P A P W P |
| 836 | cgctgcattatcagccactcctcttcactcctttctatggctcca |
| | R C I I S H S S S L L S M A P |
| 881 | gaagtgaagagctctgaaatgcctaaattgccagatgggaacatc |
| | E V K S S E M P K L P D G N I |
| 926 | tttactgcttggagaaagccacacaggagttacctgacctgcctt |
| | F T A W R K P H R S Y L T C L |
| 971 | ggatggtga 979 |
| | G W * |

CT1.29 (CTSP-9)
(SEQ ID NO. 33)

| | |
|---|---|
| 287 | atgtcatcagtttcaactgcttttgcactactaagacgcttttct |
| | M S S V S T A F A L L R R F S |
| 332 | gggttaattagcagcaatacagacaacgatcttttattcaacaac |
| | G L I S S N T D N D L L F N N |
| 377 | ctctctcgagatattttaaataatttctcacactcgaaaaacatg |
| | L S R D I L N N F S H S K N M |
| 422 | cagaagcgactattggcaaacctgaagagggtggaataccaaatg |
| | Q K R L L A N L K R V E Y Q M |
| 467 | gctgaactggaatattttctagttagcgagggtttgagaggtgcg |
| | A E L E Y F L V S E G L R G A |
| 512 | tcaggtctccagaaattcacctcaaaagcgtacaggatgtaa 553 |
| | S G L Q K F T S K A Y R M * |

### Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety, and particularly for the purposed cited herein.

What is claimed is:

1. An isolated nucleic acid molecule selected from the group consisting of:
(a) complements of nucleic acid molecules that hybridize under high stringency conditions to a second nucleic acid molecule comprising a nucleotide sequence set forth as any of SEQ ID NOs: 24-28,
(b) nucleic acid molecules that differ from the nucleic acid molecules of (a) in codon sequence due to the degeneracy of the genetic code, and
(c) full-length complements of (a) or (b).

2. The isolated nucleic acid molecule of claim 1, wherein the isolated nucleic acid molecule comprises or consists of a nucleotide sequence set forth as any of SEQ ID NOs: 24-28, a protein-coding portion thereof, or an alternatively spliced product thereof; or a nucleotide sequence that is at least about 90%, 95%, 97%, 98% or 99% identical to a nucleotide sequence set forth as any of SEQ ID NOs: 24-28, or a full-length complement thereof.

3-8. (canceled)

9. A composition comprising the isolated nucleic acid molecule of claim 1 and a carrier, or the isolated nucleic acid molecule of claim 1 attached to a solid substrate.

10. (canceled)

11. A kit comprising:
one or more nucleic acid molecules that hybridize under high stringency conditions to a nucleic acid molecule of claim 1 (CTSP-6), CT1.19 (CTSP-7; SEQ ID NO:34), or CT1.26 (CTSP-8; SEQ ID NO:35), optionally wherein the one or more nucleic acid molecules are detectably labeled, optionally wherein the one or more nucleic acids are bound to a solid substrate.

12. The kit of claim 11, further comprising a nucleic acid molecule that hybridizes under high stringency conditions to a nucleic acid molecule that encodes CTSP-5 (SEQ ID NO:36).

13. (canceled)

14. The kit of claim 11, wherein the one or more nucleic acid molecules consist of a first primer and a second primer, wherein the first primer and the second primer are constructed and arranged to selectively amplify at least a portion of a nucleic acid molecule encoding CT1.11 (CTSP-6; SEQ ID NOs:24-27), CT1.19 (CTSP-7; SEQ ID NO:34), CT1.26 (CTSP-8; SEQ ID NO:35), or CT1.29 (CTSP-9; SEQ ID NO:28).

15. The kit of claim 14, further comprising an additional primer pairs, a first primer and a second primer, to selectively amplify at least a portion of a nucleic acid molecule encoding CT1.1 (CTSP-5; SEQ ID NO:36).

16. (canceled)

17. An expression vector comprising the isolated nucleic acid molecule of claim 1 operably linked to a promoter.

18. An isolated host cell transformed or transfected with the expression vector of claim 17.

19. An isolated host cell transformed or transfected with an expression vector comprising an isolated nucleic acid molecule encoding CT1.1 (CTSP-5; SEQ ID NO:36), CT1.19 (CTSP-7; SEQ ID NO:34), or CT1.26 (CTSP-8; SEQ ID NO:35), optionally wherein the host cell is a dendritic cell.

20. The isolated host cell of claim 18, wherein the host cell expresses a MHC molecule, optionally wherein the host cell expresses the MHC molecule recombinantly, optionally wherein the host cell is a dendritic cell.

21-22. (canceled)

23. A composition comprising the isolated host cell of claim 18 and a carrier.

24. An isolated polypeptide encoded by the isolated nucleic acid molecule of claim 1, or a fragment thereof that is at least eight amino acids in length.

25. (canceled)

26. An isolated polypeptide encoded by the CT1.19 (CTSP-7; SEQ ID NO:34) or CT1.26 (CTSP-8; SEQ ID NO:35) gene, or a fragment thereof that is at least eight amino acids in length.

27-32. (canceled)

33. An isolated antibody or antigen-binding fragment thereof that selectively binds to the isolated polypeptide of claim 24, optionally wherein the antibody is a monoclonal antibody, a human antibody, a domain antibody, a humanized antibody, a single chain antibody or a chimeric antibody, wherein the antibody fragment is a F(ab′)₂, Fab, Fd, or Fv fragment.

34-40. (canceled)

41. A method of diagnosing cancer in a subject comprising:
obtaining a biological sample from the subject, and
determining the presence in the biological sample of an antibody that binds specifically to one or more polypeptides encoded by the isolated nucleic acid molecule of claim 1 or encoded by the CT1.19 (CTSP-7) (SEQ ID NO. 34) or CT1.26 (CTSP-8) (SEQ ID NO. 35) gene, wherein the presence of the antibody is indicative of the subject having cancer,
optionally wherein the step of determining the presence of the antibody comprises: contacting the biological sample with one or more polypeptides encoded by a nucleic acid molecule comprising (1) a nucleotide sequence set forth as any of SEQ ID NOs: 24-28 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1), and determining the binding of the antibody to the polypeptide,
optionally wherein the polypeptide comprises an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length.

42-48. (canceled)

49. A method for diagnosing cancer in a subject comprising:
obtaining a biological sample from a subject, and
determining the expression in the biological sample of a polypeptide or a nucleic acid molecule that encodes the polypeptide, wherein the nucleic acid molecule comprises (1) a nucleotide sequence of the isolated nucleic acid molecule of claim 1 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1),
wherein the expression in the biological sample of the polypeptide or the nucleic acid molecule that encodes it is indicative of the subject having cancer,
optionally wherein the polypeptide comprises an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length,
optionally wherein the step of determining the expression of the polypeptide or the nucleic acid molecule that encodes the polypeptide comprises contacting the biological sample with an agent that selectively binds to the polypeptide or the nucleic acid molecule that encodes the polypeptide.

50-63. (canceled)

64. A method for determining onset, progression, or regression of cancer in a subject comprising:
obtaining from a subject a first biological sample at a first time,
determining the expression in the first sample of a polypeptide or a nucleic acid molecule that encodes the polypeptide, wherein the nucleic acid molecule comprises (1) a nucleotide sequence of the isolated nucleic acid molecule of claim 1 or the nucleotide sequence of the CT1.19 (CTSP-7) (SEQ ID NO. 34) or CT1.26 (CTSP-8) (SEQ ID NO. 35) gene, or (2) a nucleotide sequence that is at least 90% identical to the nucleotide sequence of (1),
obtaining from the subject a second biological sample at a second time subsequent to the first time,
determining the expression in the second sample of the polypeptide or the nucleic acid molecule that encodes the polypeptide, and
comparing the expression in the first sample to the expression in the second sample as a determination of the onset, progression, or regression of the cancer,
wherein an increase in expression in the second sample compared to the first sample is indicative of onset or progression of the cancer, and wherein a decrease in the expression in the second sample compared to the first sample is indicative of regression of the cancer,
optionally wherein the polypeptide comprises an amino acid sequence set forth as any of SEQ ID NOs: 29-33, or an amino acid sequence corresponding to the CT1.19 (CTSP-7) (SEQ ID NO. 34) or CT1.26 (CTSP-8) (SEQ ID NO. 35) gene, or a fragment thereof that is at least eight amino acids in length,
optionally wherein the step of determining the expression of the polypeptide or the nucleic acid molecule that encodes the polypeptide comprises contacting the first biological sample and the second biological sample with an agent that selectively binds to the polypeptide or the nucleic acid molecule that encodes the polypeptide.

65-78. (canceled)

79. A method for treating cancer in a subject, comprising:
administering to the subject an agent that stimulates an immune response to a polypeptide encoded by a nucleic acid molecule comprising a nucleotide sequence that is at least 90% identical to the isolated nucleic acid molecule of claim 1 or a nucleotide sequence of CT1.19 (CTSP-7) (SEQ ID NO. 34) or CT1.26 (CTSP-8) (SEQ ID NO. 35), optionally wherein the agent that stimulates the immune response is the polypeptide, a cell that expresses the polypeptide, a peptide fragment of the polypeptide, or a complex of a peptide fragment of the polypeptide and a MHC molecule.

80-92. (canceled)

93. A method for treating cancer in a subject comprising:
administering to a subject an effective amount of an antibody or antigen-binding fragment thereof that specifically binds to a polypeptide that comprises an amino acid sequence encoded by the isolated nucleic acid molecule of claim 1, or an amino acid sequence corresponding to the CT1.19 (CTSP-7) (SEQ ID NO. 34) or CT1.26 (CTSP-8) (SEQ ID NO. 35) gene, or a peptide fragment thereof, or a complex of the peptide fragment and a MHC or HLA molecule.

94-105. (canceled)
